(19) United States
(12) Patent Application Publication
(10) Pub. No.: US 2004/0219138 A1
(43) Pub. Date: Nov. 4, 2004

(54) Combinations of superoxide dismutase mimetics and opioids

(75) Inventor: Daniela Salvemini, Chesterfield, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignees: MetaPhore Pharmaceuticals, Inc.; Pharmacia Corporation

(21) Appl. No.: 10/738,578

(22) Filed: Dec. 16, 2003

Related U.S. Application Data
(63) Continuation of application No. 09/997,974, filed on Nov. 30, 2001.
Application No. 09/997,974 is a continuation-in-part of application No. 09/634,152, filed on Aug. 9, 2000, now Pat. No. 6,395,725.
Application No. 09/634,152 is a division of application No. 09/057,831, filed on Apr. 9, 1998, now Pat. No. 6,180,620.
(60) Provisional application No. 60/050,402, filed on Jun. 20, 1997.

(51) Int. Cl. A61K 38/44; A61K 31/555; A61K 31/485

(52) U.S. Cl. 424/94.4; 514/184; 514/282

(57) ABSTRACT

Combinations of synthetic low molecular weight catalysts for the dismutation of superoxide and opioids are potent analgesics that are effective in elevating the pain threshold in hyperalgesic conditions.

### CROSS-REFERENCES TO RELATED APPLICATIONS

This application is a continuation of co-pending U.S. application Ser. No. 09/997,974 filed Nov. 30, 2001 which is a continuation-in-part of U.S. application Ser. No. 09/634,152 filed Aug. 9, 2000, now U.S. Pat. No. 6,395,725, which is a divisional of U.S. application Ser. No. 09/057,831 filed April 9,1998, now U.S. Pat. No. 6,180,620, which claimed the benefit of U.S. Provisional Application No. 60/050,402 filed Jun. 20, 1997. Each patent and patent application above is incorporated herein by reference in its entirety.

### STATEMENT REGARDING FEDERALLY SPONSORED RESEARCH OR DEVELOPMENT

Not Applicable.

### REFERENCE TO A SEQUENCE LISTING

Not Applicable.

### BACKGROUND OF THE INVENTION

1. Field of the Invention

The present invention relates to the treatment of humans and lower animals in pain management: to prevent or relieve pain, to prevent or reverse tolerance to opioid analgesics and hyperalgesia associated with prolonged opioid treatment, and to prevent or reduce symptoms of opioid withdrawal and related withdrawal syndromes.

2. Description of the Related Art

Numerous analgesics are known to medical science. Many analgesics fall into one of two large categories—nonsteroidal analgesic/anti-inflammatory drugs (NSAIDs) and opioids. NSAIDs operate by inhibiting cyclooxygenase enzymes (including cyclooxygenase-1 and cyclooxygenase-2, also known as COX-1 and COX-2 respectively) and thereby the synthesis of prostaglandins. Prostaglandins sensitize pain receptors, lowering the pain threshold and making normal stimuli, such as touch and stretch sensations, painful. NSAIDs can be quite effective at returning the lowered pain threshold to normal but do not elevate the pain threshold. Common NSAIDs available over-the-counter include: ibuprofen (Advil®), naproxen (Aleve® or Naprosyn®)), and aspirin (Bayer®). Prescription NSAIDs include: celecoxib—Celebrex(®, diclofenac—Voltaren®, etodolac—Lodine®, fenoprofen—Nalfon®, indomethacin—Indocin®, ketoprofen—Orudis®, Oruvail®, ketoralac—Toradol®, oxaprozin—Daypro®, nabumetone—Relafen®, sulindac—Clinoril®, tolmetin—Tolectin®, and rofecoxib—Vioxx®).

A second class of pain relievers, opioids, operate by mimicking natural peptides such as enkephalins and endorphins to stimulate one or more of the μ-, δ- and κ-receptor systems in the nervous system. Opioids elevate the pain threshold so that normally painful stimuli are perceived as less painful or even euphoric. Opioids are commonly used in the clinical management of severe pain, including chronic severe pain of the kind experienced by cancer patients. Common opioids include morphine, oxycontin, oxycodone, codeine and fentanyl.

Capsaicin and its derivatives operate by depleting local stores of substance P, a neuropeptide involved in the transmission of pain impulses and are used in several OTC analgesic products.

Each of these classes of compounds has inherent problems and limitations. The opioid analgesics are antagonized by analogous N-allyl compounds such as naloxone; the NSAID analgesics are not. NSAIDs that are nonselective for the cyclooxygenase-2 produced in inflammation (COX-2) also inhibit constitutive cyclooxygenase-1 (COX-1), causing undesirable damage to the gastric mucosa. They have limited effectiveness as analgesics in lowering an elevated threshold to normal and are generally used for mild to moderate pain. They are also ineffective drugs for elevation of the pain threshold above normal levels, which prevents their use in pain such as surgical pain where an underlying pathological condition has not elevated the pain threshold.

Opioids have problems with tolerance and dependency, so that over a course of therapy increasing dosages of compound are required to achieve the same level of analgesia, and cessation of opioid administration when analgesia is no longer needed elicits a withdrawal syndrome with unpleasant and potentially serious symptoms. The dependency and withdrawal syndrome both make it difficult for the clinician to discontinue opioid therapy even when the opioids are no longer effective in relieving pain because of the development of tolerance. Narcotic induced hyperalgesia (NIH) can also develop in association with tolerance to the opioids. All of these factors limit the usefulness of opioids in the management of chronic severe pain, despite their potency.

No adequate strategy has been devised to overcome the development of opioid tolerance and provide an ongoing approach to the management of chronic severe pain. Mechanisms of tolerance are not well understood but are known to involve the NMDA receptor, since the NMDA receptor antagonist MK-801 has been shown in rats to prevent morphine tolerance. NMDA stimulates nitric oxide synthase (NOS) and NOS has been observed histochemically in tissues that contain opioid receptors and are important in the pain response, such as the amygdala, cortical gray matter, and the substantia gelatinosa of the spinal cord. Non-selective NOS inhibitors such as NG-nitroarginine prevent and reverse morphine tolerance. However, nonselective inhibition of NOS is associated with a vast array of undesirable side effects, including hypertension, increased platelet and white blood cell reactivity, decreased cerebral blood flow, and gastrointestinal and renal toxicity.

Capsaicin and some of its derivatives, in addition to producing analgesia, also elicit a burning sensation. This effect is responsible for the pungency of hot peppers (Capscum spp.) and limits the applicability of many members of this series of compounds.

For these and other reasons, a continuing need exists for new high potency analgesics which do not result in the drawbacks listed above. A need also exists for methods for reversing tolerance to opioid analgesics so that patients who require these drugs for pain over extended periods can do so without loss of potency and efficacy.

### BRIEF SUMMARY OF THE INVENTION

Accordingly, it is an object of the invention to overcome these and other problems associated with the related art. These and other objects, features and technical advantages are achieved by providing combinations of opioids and synthetic superoxide dismutase catalysts for treating, preventing, reversing or inhibiting pain or inflammation when administered to a patient in need thereof.

This invention provides a combination of compositions comprising (a) at least one opioid; and (b) at least one synthetic superoxide dismutase catalyst. In one aspect, the combination is capable of treating, preventing, reversing or inhibiting pain or inflammation when administered to a patient in need thereof. In one embodiment, the combination is capable of producing an additive or synergistic antihyperalgesia or antinociception effect in the patient after administering the combination.

Preferably, the opioid of the combination comprises at least about 50% less than the same opioid administered alone to achieve the antihyperalgesia or antinociception effect. More preferably, the opioid of the combination comprises at least about 25% less than the same opioid administered alone to achieve the antihyperalgesia or antinociception effect. Still more preferably, the opioid of the combination comprises at least about 10% less than the same opioid administered alone to achieve the antihyperalgesia or antinociception effect. And still more preferably, the opioid of the combination comprises at least about 1% less than the same opioid administered alone to achieve the antihyperalgesia or antinociception effect.

In accordance with one aspect of the invention, the opioid and the synthetic superoxide dismutase catalyst are combined prior to administration to the patient. In another aspect, the opioid and the synthetic superoxide dismutase catalyst are combined upon administration to the patient.

Preferably, the opioid is selected from the group consisting of morphine, oxycontin, oxycodone, codeine, fentanyl and any combination thereof. In accordance with another aspect of the invention, the synthetic superoxide dismutase catalyst is represented by the formula:

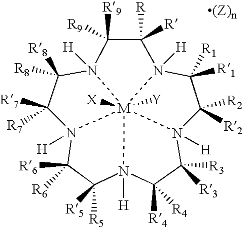

wherein (a) R, R′, R₁, R′₁, R₂, R′₂, R₃, R′₃, R₄, R′₄, R₅, R′₅, R₆, R′₆, R₇, R′₇, R₈, R′₈, R₉, and R′₉ independently are selected from the group consisting of hydrogen and substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and (b) optionally, R₁ or R′₁ and R₂ or R′₂, R₃ or R′₃ and R₄ or R′₄, R₅ or R′₅ and R₆ or R′₆, R₇ or R′₇, and R₈, or R′₈, or R₉ or R′₉ and R or R′ together with the carbon atoms to which they are attached independently form a substituted or unsubstituted, saturated, partially saturated or unsaturated cyclic or heterocyclic having 3 to 20 carbon atoms; and (c) optionally, R or R′ and R₁ or R′₁, R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle wherein the hydrogen attached to the nitrogen which is both part of the heterocycle and the macrocycle and the R groups attached to the carbon atoms which are both part of the heterocycle and the macrocycle are absent; and (d) optionally, R and R′, R₁ and R′₁, R₂ and R′₂, R₃ and R′₃, R₄ and R′₄, R₅ and R′₅, R₆ and R′₆, R₇, and R′₇, R₈, and R′₈, and R₉ and R′₉, together with the carbon atom to which they are attached independently form a substituted or unsubstituted, saturated, partially saturated, or unsaturated cyclic or heterocyclic having 3 to 20 carbon atoms; and (e) optionally, one of R, R′, R₁, R′₁, R₂, R′₂, R₃, R′₃, R₄, R′₄, R₅, R′₅, R₆, R′₆, R₇, R′₇, R₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉attached to a different carbon atom in the macrocycle are bound to form a strap represented by the formula:

—(CH₂)ₓ—M—(CH₂)_{w}—L—(CH₂)_{z}—J—(CH₂)_{y—}

wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and (f) combinations of any of (a) through (e) above; and wherein M is selected from the group consisting of copper, manganese and zinc; X, Y and Z are pharmaceutically acceptable counter ions, or together are a pharmaceutically acceptable polydentate ligand; and n is an integer from 0 to 3.

Preferably, the synthetic superoxide dismutase catalyst is represented by the formula:

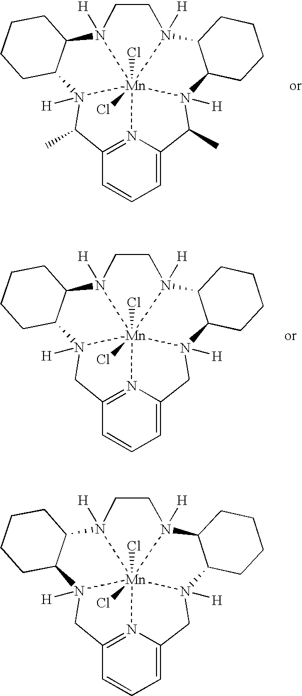

This invention provides a compound of the formula Aₙ-Qₘ, wherein A is a superoxide dismutase catalyst moiety, Q is an opioid moiety, and n and m are independently integers from 1 to 3. Preferably, the opioid is selected from the group consisting of morphine, oxycontin, oxycodone, codeine, fentanyl and any combination thereof. In accordance with one aspect of the invention, the synthetic superoxide dismutase catalyst moiety is represented by the formula:

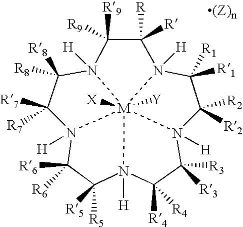

wherein (a) R, R′, R₁, R′₁, R₂, R′₂, R₃, R′₃, R₄, R′₄, R₅, R′₅, R₆, R′₆, R₇, R′₇, R₈, R′₈, R₉, and R′₉, independently are selected from the group consisting of hydrogen and substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and (b) optionally, R₁ or R′₁ and R₂ or R′₂, R₃ or R′₃ and R₄ or R′₄, R₅ or R′₅ and R₆ or R′₆, R₇ or R′₇ and R₈ or R′₈, or R₉ or R′₉ and R or R′ together with the carbon atoms to which they are attached independently form a substituted or unsubstituted, saturated, partially saturated or unsaturated cyclic or heterocyclic having 3 to 20 carbon atoms; and (c) optionally, R or R′and R₁ or R′₁, R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle wherein the hydrogen attached to the nitrogen which is both part of the heterocycle and the macrocycle and the R groups attached to the carbon atoms which are both part of the heterocycle and the macrocycle are absent; and (d) optionally, R and R′, R₁ and R′₁, R₂ and R′₂, R₃ and R′₃, R₄ and R′₄, R₅ and R′₅, R₆ and R′₆, R₇, R₈ and R′₈, and R₉ and R′₉, together with the carbon atom to which they are attached independently form a substituted or unsubstituted, saturated, partially saturated, or unsaturated cyclic or heterocyclic having 3 to 20 carbon atoms; and (e) optionally, one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇R₈, R′₈, R₉, and R′₉ attached to a different carbon atom in the macrocycle are bound to form a strap represented by the formula:

—(CH₂)ₓ—M—(CH₂)_{w}—L—(CH₂)_{z}—J—(CH₂)_{y—}

wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and (f) combinations of any of (a) through (e) above; and wherein M is selected from the group consisting of copper, manganese and zinc; X, Y and Z are pharmaceutically acceptable counter ions, or together are a pharmaceutically acceptable polydentate ligand; and n is an integer from 0 to 3.

Preferably, the synthetic superoxide dismutase catalyst is represented by the formula:

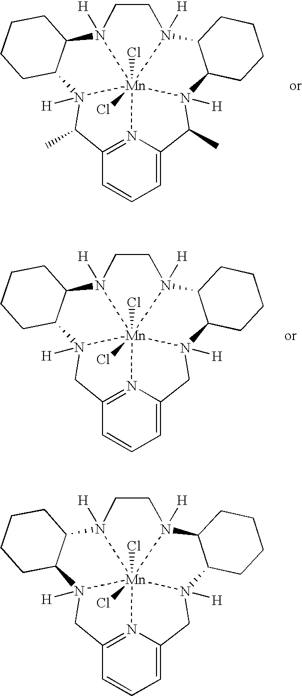

These and other features, aspects and advantages of the present invention will become better understood with reference to the following description, examples and appended claims.

### BRIEF DESCRIPTION OF THE SEVERAL VIEWS OF THE DRAWINGS

FIG. 1 is a graph depicting the results of a study on the inhibition of carrageenan-induced hyperalgesia by intravenously injected SC-72325. The drug was given at 3 hours post carrageenan injection.

FIGS. 2 and 3 are graphs depicting the results of a study on inhibition of carrageenan-induced hyperalgesia by intramuscular injection of either SOD mimic compound SC-72325 (Example 157) or the nonsteroidal anti-inflammatory drug ketorolac.

FIG. 4 is a graph depicting the results of a study comparing the effects of SC-72325 versus ketorolac on carrageenan-induced increase of PGE-2 in cerebrospinal fluid.

FIG. 5 is a graph depicting the results of a study comparing the effects of SC-72325 versus ketorolac on carrageenan-induced release of PGE-2 in paw exudate.

FIG. 6 is a graph depicting the results of a study on inhibition of formalin-induced nociception by subcutaneous injection of SC-72325A.

FIG. 7 is a graph depicting the results of a study on inhibition of carrageenan-induced hyperalgesia by subcutaneous injection of SC-72325A. The drug was given at three (3) hours post carrageenan.

FIG. 8 is a graph depicting the results of a study on inhibition of carrageenan-induced hyperalgesia by subcutaneous injection of SC-72325A and morphine-SC-72325A synergistic effect.

FIG. 9 is a graph depicting the results of a study on carrageenan-induced hyperalgesia by SC-72325A and ketorolac. Drugs given by subcutaneous injection at three (3) hours post carrageenan.

FIG. 10 is a graph depicting the results of a study on the time-related and dose-dependent antihyperalgesia effect of SC-72325A over the dose range of 0.3 to 30 mg/kg in the SNL (L₅/L₆) model. Drugs administered via subcutaneous injection.

FIG. 11 is a graph depicting the results of a study on the time-related and dose-dependent attenuation of cold allodynia of SC-72325A over the dose range of 1 to 10 mg/kg.

### DETAILED DESCRIPTION OF THE INVENTION

This invention is based upon surprising discoveries involving certain organometallic complexes designed as synthetic catalysts for use in the body. These catalysts have been designed as synthetic replacements for or adjuncts to the naturally occurring enzyme superoxide dismutase (SOD).

Naturally occurring SOD scavenges and eliminates the toxicity of free superoxide radicals (O₂^{•-}) liberated by certain metabolic reactions. Although these free radicals play a major (and deleterious) role in the inflammatory response and other toxic reactions to injury, neither superoxide nor SOD has been known to be directly involved in pain perception. In addition, SOD has a very short biological half-life, on the order of seconds or minutes rather than hours, so it would be considered unsuitable for treatment of conditions in which increased dismutation of superoxide radicals would be desirable over periods of from minutes to days.

Dismutation of superoxide radicals is catalyzed by a coordinated transition metal ion. In the natural SOD enzyme, the metal is manganese, copper or zinc and the coordination complex is a conventional protein structure. Synthetic SOD catalysts also use transition metals, complexed with low molecular weight organic ligands, generally polydentate N-containing macrocycles. These molecules have been designed to be highly efficient and to overcome the pharmacokinetic disadvantages of natural SOD enzyme. The k_{cat} of some of these compounds is as high as about 10⁹ (see Example 165), indicating extraordinary catalytic efficiency, as effective as the natural enzyme and approaching the theoretical rate at which diffusion can deliver free radical substrate to the catalyst under biological conditions. They also have oil:water partition coefficients (_{log} P) that provide excellent bioavailability, and stability in the body on the order of hours to days. Their small size and low molecular weight makes it possible for the synthetic catalysts to cross membrane barriers that restrict movement of natural SOD, and their non-protein structure reduces the risk of allergic reactions that have been a problem with the administration of protein-based recombinant SOD. Finally, natural SOD produces hydrogen peroxide in the process of dismutating superoxide, yet hydrogen peroxide inhibits natural SOD, effectively self-limiting the efficacy of the natural compound. In contrast, synthetic small-molecule SOD catalysts are not susceptible to the action of hydrogen peroxide and thus retain their effectiveness.

Synthetic SOD catalysts have been proposed in the past for the treatment and prevention of inflammation, ischemia-reperfusion injury, and similar conditions where tissue damage is mediated by levels of free superoxide radicals that overwhelm natural SOD, but they have not been proposed for use as analgesics in the treatment of pain.

It has now been discovered that synthetic SOD catalysts are highly effective as analgesics to prevent or provide relief from pain in conditions in which the pain threshold is elevated. It has also been discovered that these same compounds are effective in preventing or reversing tolerance to opioid analgesics, that are used to elevate the pain threshold above normal levels.

No known mechanism accounts for the analgesic properties of these compounds. However, the data shown in the examples illustrate that these compounds can be as effective as morphine in preventing and relieving certain kinds of pain. Y. Lin et al., Int. J. Maxillofac. Surg. 23:428429 (1994) reported the use of intra-articular injections of human Cu/Zn superoxide dismutase as a nonsteroidal anti-inflammatory in the treatment of temporomandibular joint dysfunction. Positive response in terms of mandibular movement and pain was observed in 83% of patients. The authors note that the results “are remarkable because SOD has been studied and shown to exert no peripheral or central analgesic effect.” They attribute the reduction in pain to the reduction in tissue injury and inflammation associated with TMJ dysfunction.

Similarly, no known mechanism accounts for the ability of these compounds to prevent or reverse tolerance to opioids. G. I. Elmer et al., Euro. J. Pharmacol. 283 (1995) 227-232, reported that transgenic mice expressing the human Cu/Zn superoxide dismutase gene had an increase in μ-opioid receptor concentration in dopaminergic related tissues and the central grey area of the CNS, which was associated with a dose-related increased sensitivity to μ-receptor agonists such as morphine. At the same time the authors also observed conflicting effects of transgenic SOD on δ-receptor agonists (mice heterozygous for the transgene were more sensitive than homozygotes, which were more sensitive than untransformed mice) and observed no effect of transgenic SOD on κ-receptor agonists.

Superoxide dismutase activity is known to play a critical role in regulating the redox state of the cell, as reported by J. L. Cadet, Int. J. Neurosci. 40, 13 (1988). This in turn is reported by Marzullo and Hine, Science 208,1171 (1980) to significantly affect in vitro μ-and δ-opioid binding.

In particular, this invention provides a method of producing analgesia in a human or lower mammal patient, comprising administering to the patient an analgesic amount of a functional synthetic catalyst for the dismutation of superoxide radicals. Based on the data obtained, it is reasonable to expect that any superoxide dismutase catalyst will be effective in the practice of this invention. A preferred synthetic catalyst is a coordination complex of transition metal with an organic ligand. Preferred transition metals are copper, manganese and zinc. Manganese is most preferred. In general, the organic ligand is a N-containing macrocycle, and most preferred ligands are selected from the group consisting of compounds of the formula

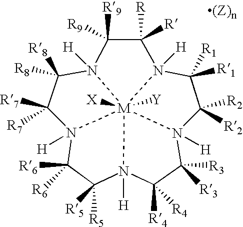

wherein R, R′, R₁, R′₁, R₂, R′₂, R₃, R′₃, R₄, R′₄, R′₅, R₆, R′₆, R₇, R′₇, R₈, R′₈, R₉ and R′₉ independently are selected from the group consisting of hydrogen and substituted or unsubstituted alkyl, alkenyl, alkynyl, cycloalkyl, cycloalkenyl, cycloalkylalkyl, cycloalkylcycloalkyl, alkylcycloalkyl, cycloalkenylalkyl, alkenylcycloalkyl, alkylcycloalkenyl, alkenylcycloalkenyl, heterocyclic, aryl and aralkyl radicals, or R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉, together with the carbon atoms to which they are attached independently form a substituted or unsubstituted saturated, partially saturated or unsaturated cyclic ring structure having 3 to 20 carbon atoms; or R or R′, R₁ or R′₁, and R₂ or R′₂, R₃ or R′₃ and R₄ or R′₄, R₅ or R′₅ and R₆ or R′₆, R₇ or R′₇, and R₈ or R′₈, and R₉ or R′₉, together with the carbon atoms to which they are attached independently form a substituted or unsubstituted nitrogen-containing heterocycle having 2 to 20 carbon atoms provided that when the nitrogen containing heterocycle is an aromatic heterocycle that does not have a hydrogen attached to the nitrogen, the hydrogen attached to the nitrogen in the macrocycle and the R groups attached to the same carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈ and R′₈ and R₉ and R′₉, together with the carbon atom to which they are attached independently form a substituted or unsubstituted saturated, partially saturated or unsaturated ring structure having 3 to 20 carbon atoms; or two of R, R′, R₁, R′₁, R₂, R′₂, R₃, R′₃, R₄, R′₄, R₅, R′₅, R₆, R′₆, R₇, R′₇, R₈, R′₈, R₉, and R′₉ attached to different carbon atoms of the macrocycle are bound to form a strap structure of the formula

—(CH₂)ₓ—M—(CH₂)_{w}—L—(CH₂)_{z}—J—(CH₂)_{y}—

wherein w, x, y and z independently are integers from 0 to 10 and M, L and J are independently selected from the group consisting of alkyl, alkenyl, alkynyl, cycloalkyl, aryl, heteroaryl, alkaryl, alkheteroaryl, aza, amido, ammonium, thio, sulfonyl, sulfinyl, sulfonamido, phosphonyl, phosphinyl, phosphino, phosphonium, keto, ester, carbamyl, ureido, thiocarbonyl, borate, borane, boraza, silyl, siloxy and silaza radicals, and combinations thereof; wherein X, Y and Z are pharmaceutically acceptable counterions or together are a pharmaceutically acceptable polydentate ligand, or are independently attached to one or more of the R groups and n is an integer from 0 to 3.

Specific examples of the above general formula are provided in the many examples below. While these specific examples provide are provided, one of skill in the art will be able to determine other variants within the scope of the above description. In addition, one of skill in the art will be able to predict and determine antianalgesic and antinociceptive effects of the other variants using the teaching of the numerous examples below.

By an “analgesic amount” of the synthetic SOD catalysts herein is meant an amount that significantly prevents or alleviates pain in the human or lower animal being treated. At a certain level stimuli are perceived as painful, while below that level they are not. This level is referred to as the pain threshold. Healthy, normal subjects exhibit a normal pain threshold that can be quantified for a given stimulus. A normal healthy individual perceives a pin prick as painful, but does not perceive the movement of a joint within its normal range of motion as painful. An individual suffering from arthritis has a lowered pain threshold and will perceive such normal movement as painful. An individual suffering from sunburn has a lowered pain threshold and may perceive the touch of a finger to be as painful as a normal individual perceives a pin prick. Because these compounds operate to elevate a lowered pain threshold, they will be effective in the treatment of such pain, and an “analgesic amount” of synthetic SOD catalysts in the treatment methods provided here also means an amount that significantly elevates the pain threshold above its pre-treatment level or prevents the pain threshold from being lowered by a pathological condition. From the standpoint of the pharmacologist and pharmaceutical scientist, this can be measured prospectively using common animal models such as the phenylquinone writhing model, the rat tail flick (radiant heat) model, the carrageenan inflammation model, the Freund's adjuvant model, and other pain models well known to pharmacological science. From the standpoint of the clinician, this can be measured according to the subjective response of each patient to a unit dose of the compound, and subsequent doses can be titrated to achieve the desired level of analgesia within the therapeutic range of the compound employed.

By an “amount sufficient to prevent or reverse tolerance to opioids” is meant The dual administration of a superoxide dismutase catalyst together with an opioid such as morphine or fentanyl allows lower doses of the morphine or fentanyl to elicit its analgesic effects while limiting its side effects. Moreover, a superoxide dismutase catalyst can reverse opioid tolerance in patients who have already developed tolerance. Thus, the superoxide dismutase catalysts restore the analgesic effect lost during prolonged treatment with an opioid. These catalysts prevent or reverse the tolerance to opioids without many of the side effects of other compounds proposed for this purpose, such as clonidine and buprenorphine. And in contrast to other proposed compounds, such as inhibitors of inducible nitric oxide synthase, the superoxide dismutase catalysts themselves have potent analgesic effects that are useful in hyperalgesic conditions such as burns, arthritis and other inflammatory diseases, migraine, and pain associated with tumor infiltration and cancer therapy.

The compounds of this invention are also useful as adjuncts in the prevention and treatment of pain with opioid analgesics, nitric oxide donors or nonsteroidal anti-inflammatory compounds. In preferred embodiments, the superoxide dismutase catalyst is administered conjointly with the opioid, NO₂ donor or NSAID compound. Administered in conjunction with an opioid, the superoxide dismutase catalyst potentiates the opioid and prevents development of tolerance and hyperalgesia. Administered after opioid tolerance, hyperalgesia and/or dependency have developed, the superoxide dismutase catalyst reverses the tolerance and hyperalgesia and reduces the symptoms of the withdrawal syndrome. Administered in conjunction with an NSAID compound or nitric oxide donor, the superoxide dismutase catalyst potentiates both the analgesia and the inflammatory action of the NSAID or NO₂ donor. These drug moieties can also be linked to provide bifunctional compounds of the formula Aₙ-Qₘ, wherein A is a superoxide dismutase catalyst moiety, Q is selected from nonsteroidal anti-inflammatory drug moieties, nitric oxide donor moieties and opioid analgesic drug moieties, and n and m are independently integers from 1 to 3. Depending upon the selection of A and Q, this can easily be done by substituting the NSAID or opioid moiety for one or more of counterion/ligands X, Y and Z in the preferred formula above. A simple approach to providing a combination containing a nitric oxide donor is to attach one or more nitrate or nitrite groups to the superoxide dismutase compound.

While not intending to be limited by theory, it is believed that the opioid withdrawal syndrome has many symptoms in common with the withdrawal syndromes associated with other addictive compounds and behaviors, including symptoms of withdrawal from cocaine, nicotine, and eating disorders such as anorexia and bulimia, especially the hyperreflexia and hyperalgesia associated with withdrawal. Accordingly, this invention also provides a method of preventing and treating symptoms of addition withdrawal, by administering to a patient in need of such treatment an amount of a superoxide dismutase catalyst that is safe and effective to prevent or reduce such symptoms.

A safe and effective amount of the compounds used in the practice of this invention is an amount that provides analgesia, thereby alleviating or preventing the pain being treated at a reasonable benefit/risk ratio as is intended with any medical treatment. In using the compounds for the reversal of opioid tolerance or reduction of withdrawal symptoms, these endpoints are used rather than analgesia. Obviously, the amount of catalyst used will vary with such factors as the particular condition that is being treated, the severity of the condition, the duration of the treatment, the physical condition of the patient, the nature of concurrent therapy (if any), the route of administration, the specific formulation and carrier employed, and the solubility and concentration of catalyst therein.

By “systemic administration” is meant the introduction of the catalyst or composition containing the catalyst into the tissues of the body, other than by topical application. Systemic administration thus includes, without limitation, oral and parenteral administration.

Depending upon the particular route of administration, and compatibility with the active compound chosen, a variety of pharmaceutically-acceptable carriers, well-known in the art, may be used. These include solid or liquid filler, diluents, hydrotropes, excipients, surface-active agents, and encapsulating substances. The amount of the carrier employed in conjunction with the catalyst is sufficient to provide a practical quantity of material per unit dose.

Pharmaceutically-acceptable carriers for systemic administration that may be incorporated into the compositions of this invention, include sugars, starches, cellulose and its derivatives, malt, gelatin, talc, calcium sulfate, vegetable oil, synthetic oils, polyols, alginic acid, phosphate buffer solutions, emulsifiers, isotonic saline, and pyrogen-free water.

The catalysts can be administered parenterally in combination with a pharmaceutically acceptable carrier such as corn oil, Cremophor EL or sterile, pyrogen-free water and a water-miscible solvent (e.g., ethyl alcohol) at a practical amount of the catalyst per dose. Preferably, the pharmaceutically-acceptable carrier, in compositions for parenteral administration, comprises at least about 90% by weight of the total composition. Parenteral administration can be by subcutaneous, intradermal, intramuscular, intrathecal, intraarticular or intravenous injection. The dosage by these modes of administration is usually in the range of from about 0.1 mg to about 20 mg per day.

Various oral dosage forms can be used, including such solid forms as tablets, capsules, granules and bulk powders. These oral forms comprise a safe and effective amount, usually at least about 5%, and preferably from about 25% to about 50% of the catalyst. Tablets can be compressed, tablet triturates, enteric-coated, sugar-coated, film-coated or multiple compressed, containing suitable binders, lubricants, diluents, disintegrating agents, coloring agents, flavoring agents, preservatives, flow-inducing agents, and melting agents. Liquid oral dosage forms include aqueous solutions, emulsions, suspensions, solutions and/or suspensions reconstituted from noneffervescent granules and effervescent preparations reconstituted from effervescent granules, containing suitable solvents, preservatives, emulsifying agents, suspending agents, diluents, sweeteners, melting agents, coloring agents, and flavoring agents. Preferred carriers for oral administration include gelatin, propylene glycol, ethyl oleate, cottonseed oil and sesame oil. Specific examples of pharmaceutically-acceptable carriers and excipients that may be used to formulate oral dosage forms containing the catalysts used in this invention, are described in U.S. Pat. No. 3,903,297, Robert, issued Sep. 2, 1975, incorporated by reference herein. Techniques and compositions for making solid oral dosage forms are described in Marshall, “Solid Oral Dosage Forms,” Modern Pharmaceutics, Vol. 7 (Banker and Rhodes, editors), 359-427 (1979), incorporated by reference herein.

By “pharmaceutically acceptable salts” is meant those salts that are safe for topical or systemic administration. These salts include the sodium, potassium, calcium, magnesium, and ammonium salts.

### EXAMPLES

Without further elaboration, it is believed that one skilled in the art can, using the preceding description, utilize the present invention to its fullest extent. The following specific examples are offered by way of illustration and not by way of limiting the remaining disclosure. Where Sprague-Dawley rats are mentioned below, 175-200g Sprague-Dawley rats were used (Harlan Sprague Dawley, Indianapolis, Ind., USA) and housed and cared for under the guidelines of the Institutional Animal Care and Use Committee. They received a subplantar injection of carrageenan (0.1 mL of a 1% suspension in 0.85% saline) into the right hind paw. At three hours post-carrageenan, when hyperalgesia is normally at a maximum, the test compound was administered intravenously at dosages of from 1-6 mg/kg. Hyperalgesia is assessed at thirty minutes to three hours post-administration of test compound.

### Example 1

### SODm Effect on Carrageenan Paw Hyperalgesia

SOD catalyst compounds were evaluated in the carrageenan hyperalgesia model described above. Results were as follows:

| Compound | Result |
|---|---|
| SC-71354 | No effect at tested dosages by intravenous injection* |
| SC-69604 | No effect at tested dosages by intravenous injection |
| SC-71449 | No effect at tested dosages by intravenous injection |
| SC-72325 | Inhibited hyperalgesia 64% at 30 minutes |
| SC-73770 | Inhibited hyperalgesia 72% at 30 minutes |

*Higher dosage levels and other routes of administration were not tested for any of the compounds.

### Example 2

### Reducing Hyperalgesia Using SODm

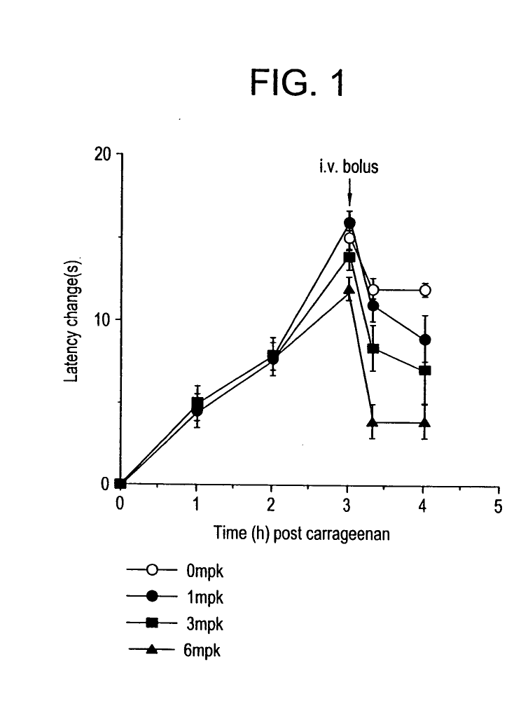

Analgesia provided by intravenous SC-72325 was evaluated over time in the carrageenan model. Results are shown in FIG. 1.

### Example 3

### Comparison of Carrageenan Paw Hyperalgesia Treatments

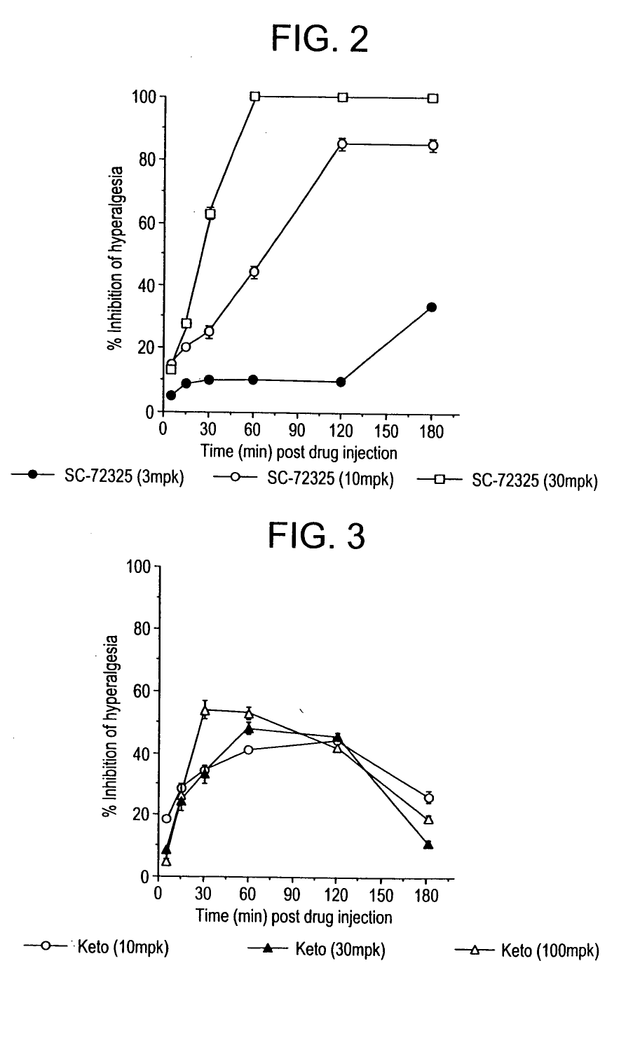

Analgesia provided by intramuscular injection of SC-72325 was evaluated over time in the carrageenan model in comparison to the anti-inflammatory drug ketorolac. Results are shown in FIGS. 2 and 3, respectively.

### Example 4

### Baseline for Carrageenan Paw Hyperalgesia Testing

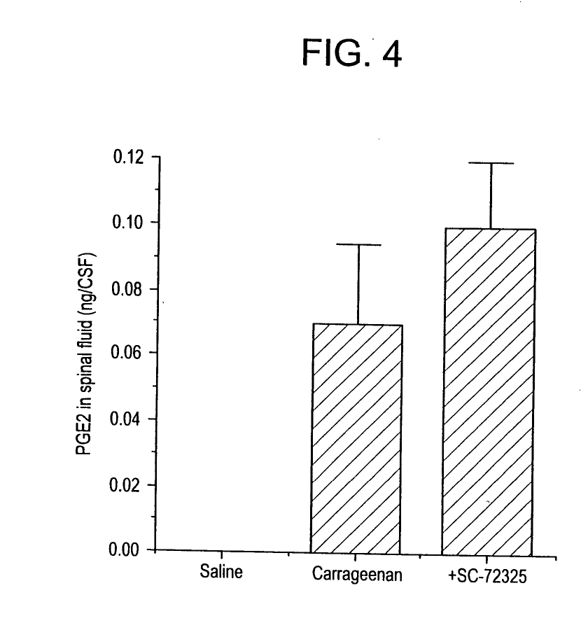

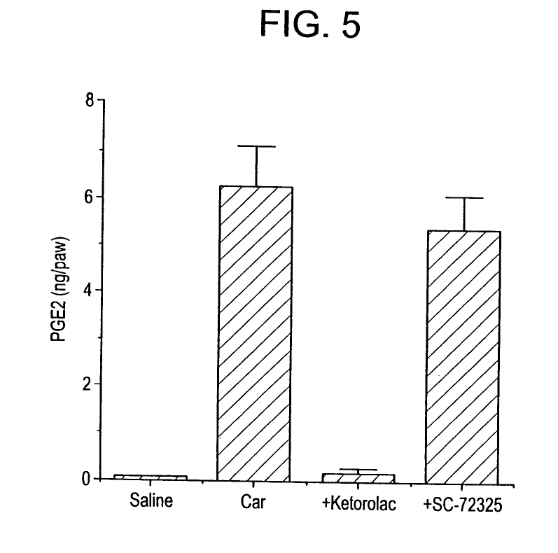

To determine whether the SOD catalyst compounds provide analgesia by some action on the prostaglandin-leukotriene system, release of prostaglandin PGE-2 was measured in rat paw exudate from the carrageenan model as well as in spinal cord fluid. Saline was used as a non-inflamed control and the anti-inflammatory ketorolac was used as a positive anti-inflammatory control. Results are shown in FIGS. 4 and 5. SC-72325 did not significantly reduce release of PGE-2 compared to the carrageenan-injected but untreated rats. Ketorolac treated rats had levels of PGE-2 release similar to non-carrageenan injected animals.

### Example 5

### Morphine Tolerance Testing

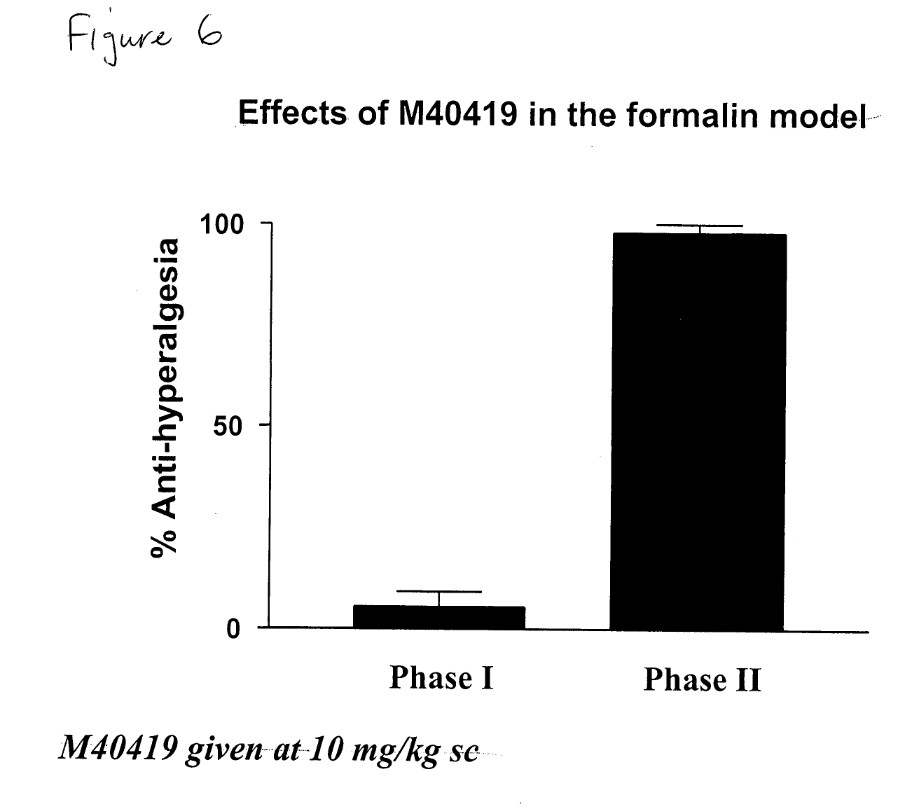

Mice were treated twice a day with either saline (naive) or morphine (s.c., 10 mg/kg) for a period of 4 days to induce tolerance. For comparison, a dose of 10 mg, or less than 0.15 mg/kg every 4 to 10 hours, is a morphine dosage routinely prescribed for the 70 kg. human adult with severe pain. On day 5, all mice received a subcutaneous challenge dose of 3 mg./kg morphine and the level of analgesia was measured 30 minutes later. Results are shown graphically in FIG. 6. Dose response measurements in normal mice have indicated that a challenge dose of 3 mg/kg would elicit 90% analgesia in naive or non-tolerant mice when assessed by the standard hot plate test. In this example, mice that were treated with morphine for 4 days showed a decreased analgesic effect from morphine on day 5 when compared with the naive mice. Tolerance to morphine was eliminated in mice that were treated with the superoxide dismutase catalyst SC-72325 administered intraperitoneally.

### Examples 6-167

The following compounds were made for use as superoxide dismutase catalysts or as ligands for combination with transition metal ions for use as superoxide dismutase catalysts within the scope of the invention. The catalytic rate constant k_{cat} is given for each compound. For k_{cat} values marked with an asterisk, the k_{cat} was measured at a pH of 8.1. For all other compounds the k_{cat} was measured at pH 7.4. Compounds marked NT were made but not tested. The ligands of Examples 11, 101, 123-135 and 138-148 were not expected to have activity without the metal ion and most were not tested. However, as can be seen by comparison of Examples 148 and 149, insertion of the metal ion into the ligand forms a complex with good superoxide dismutase activity.

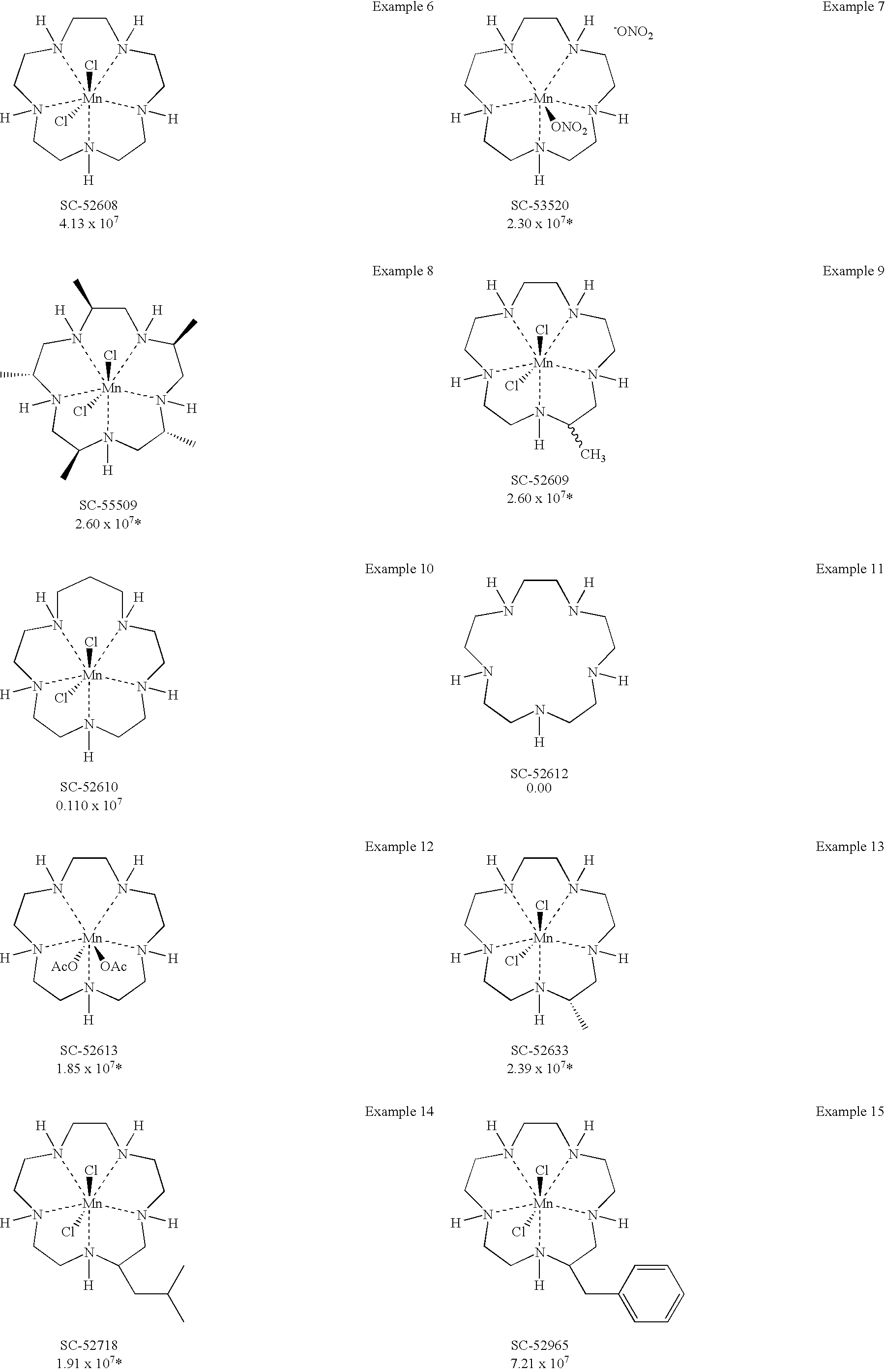

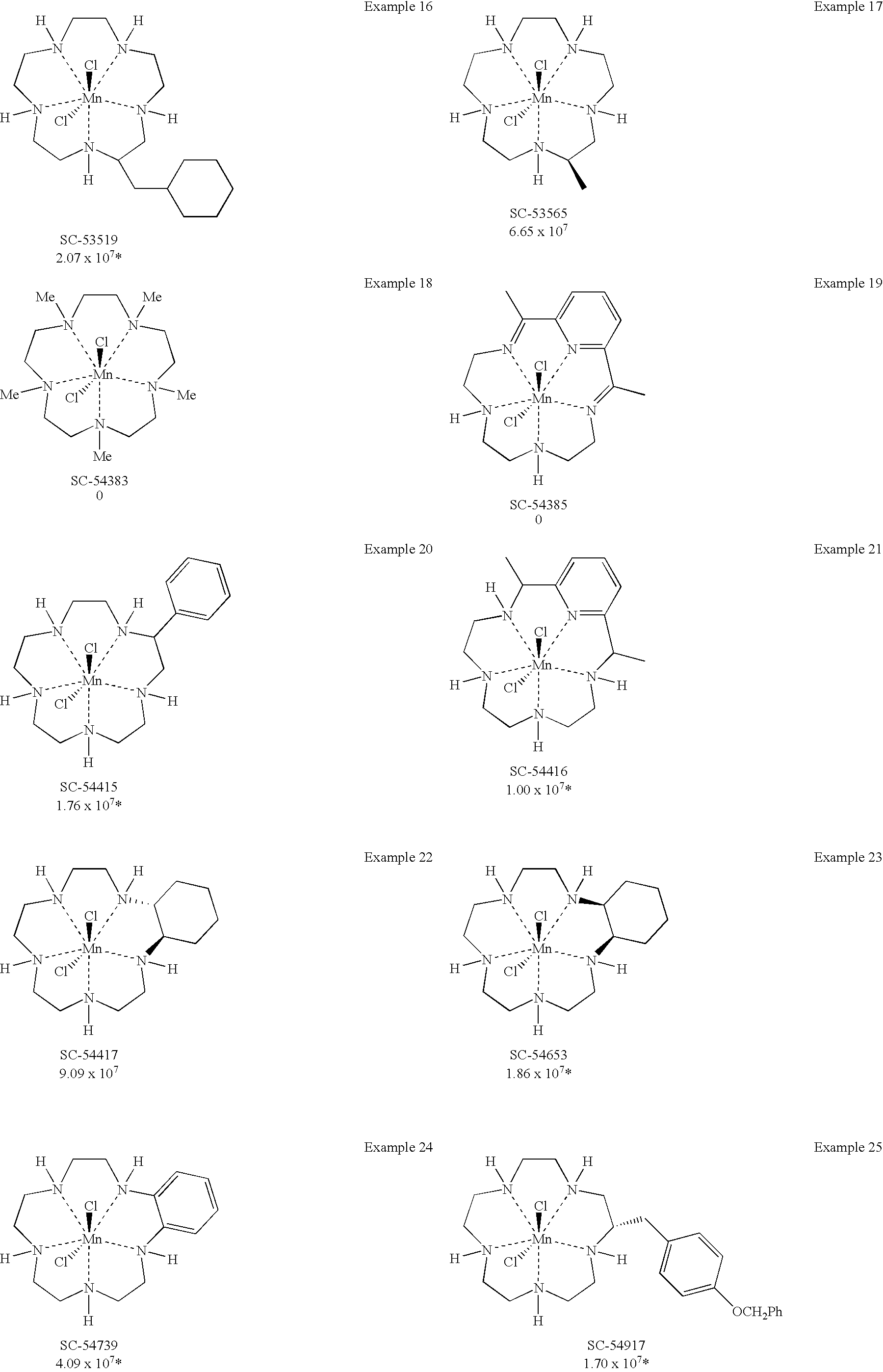

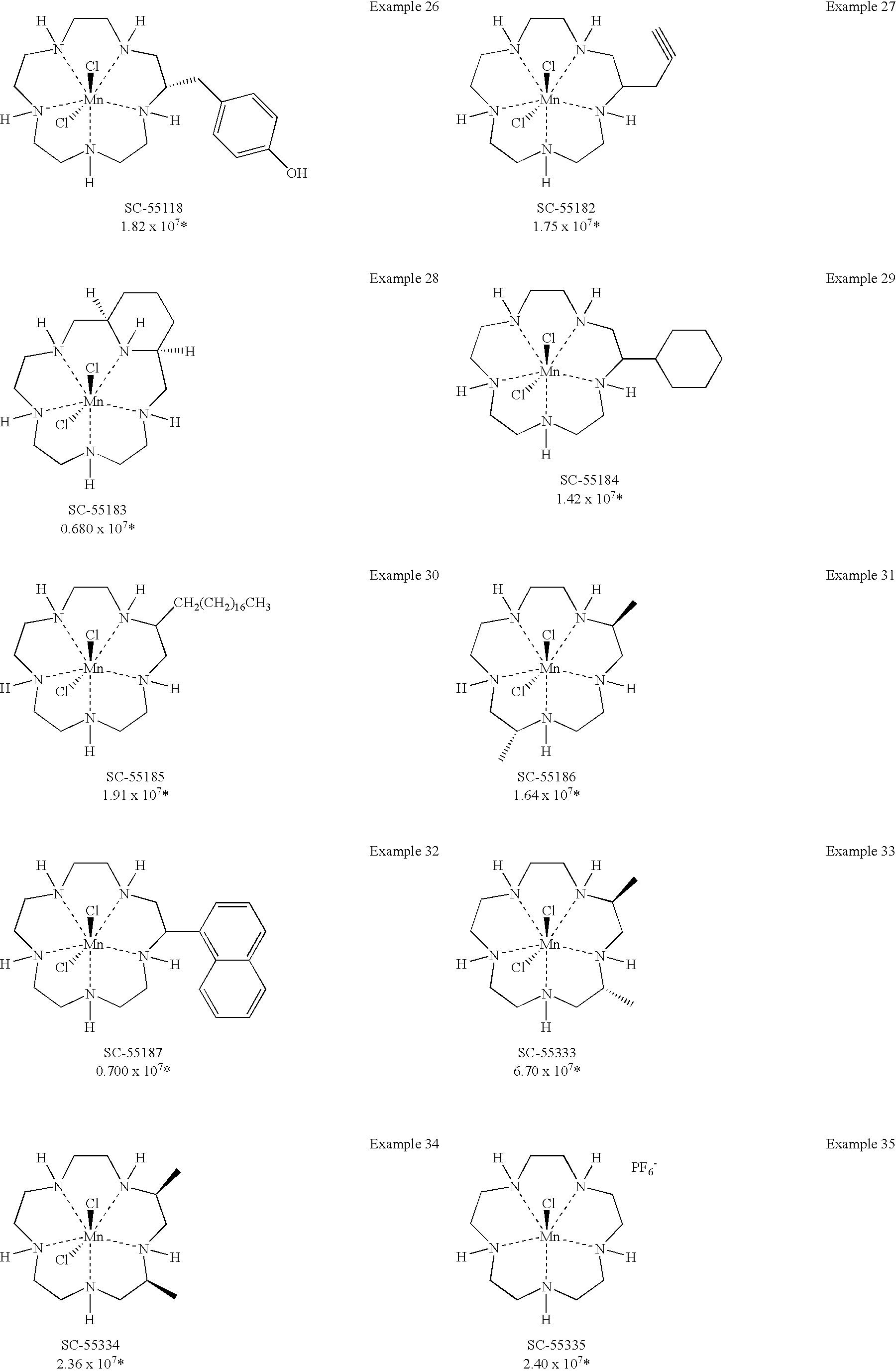

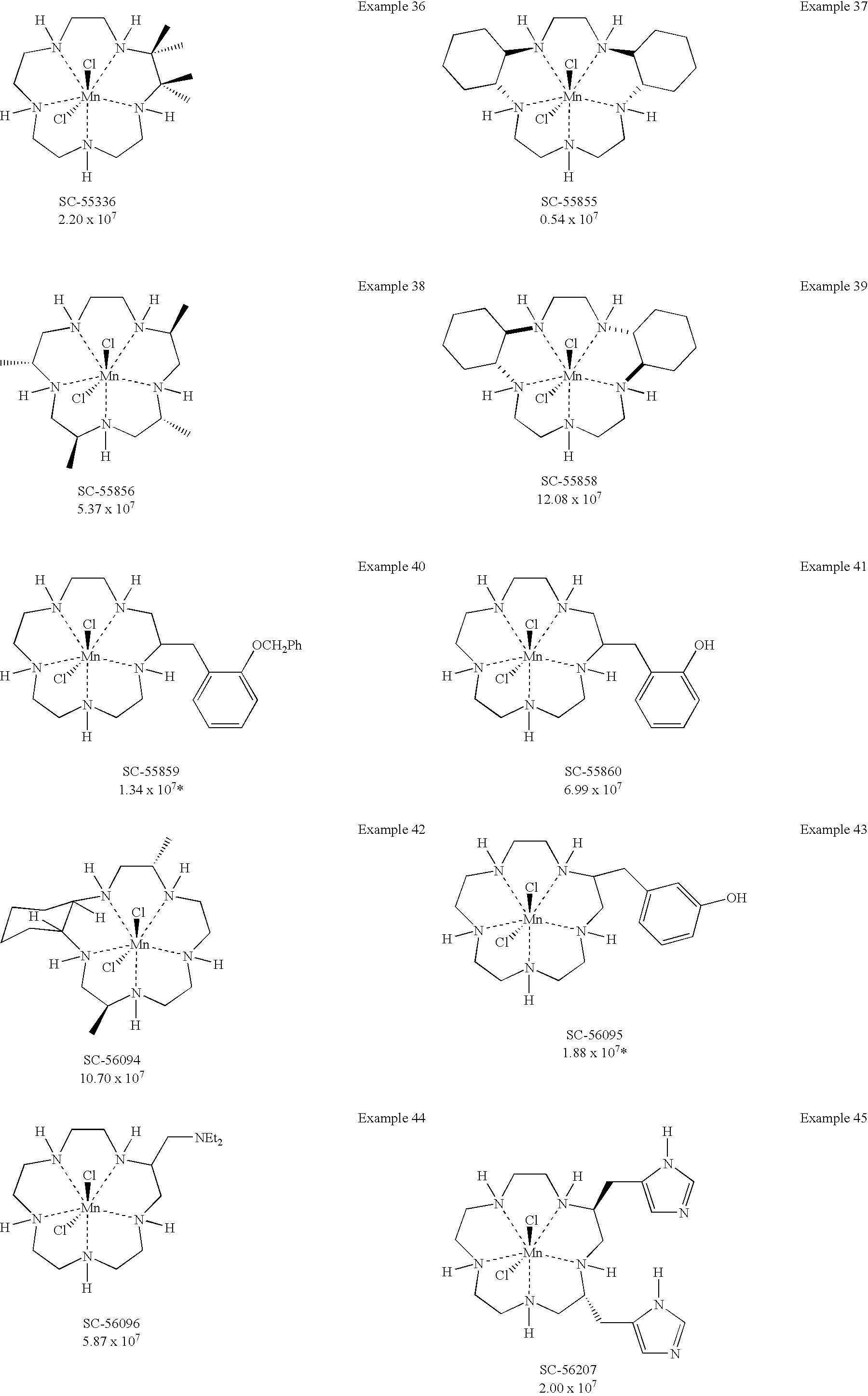

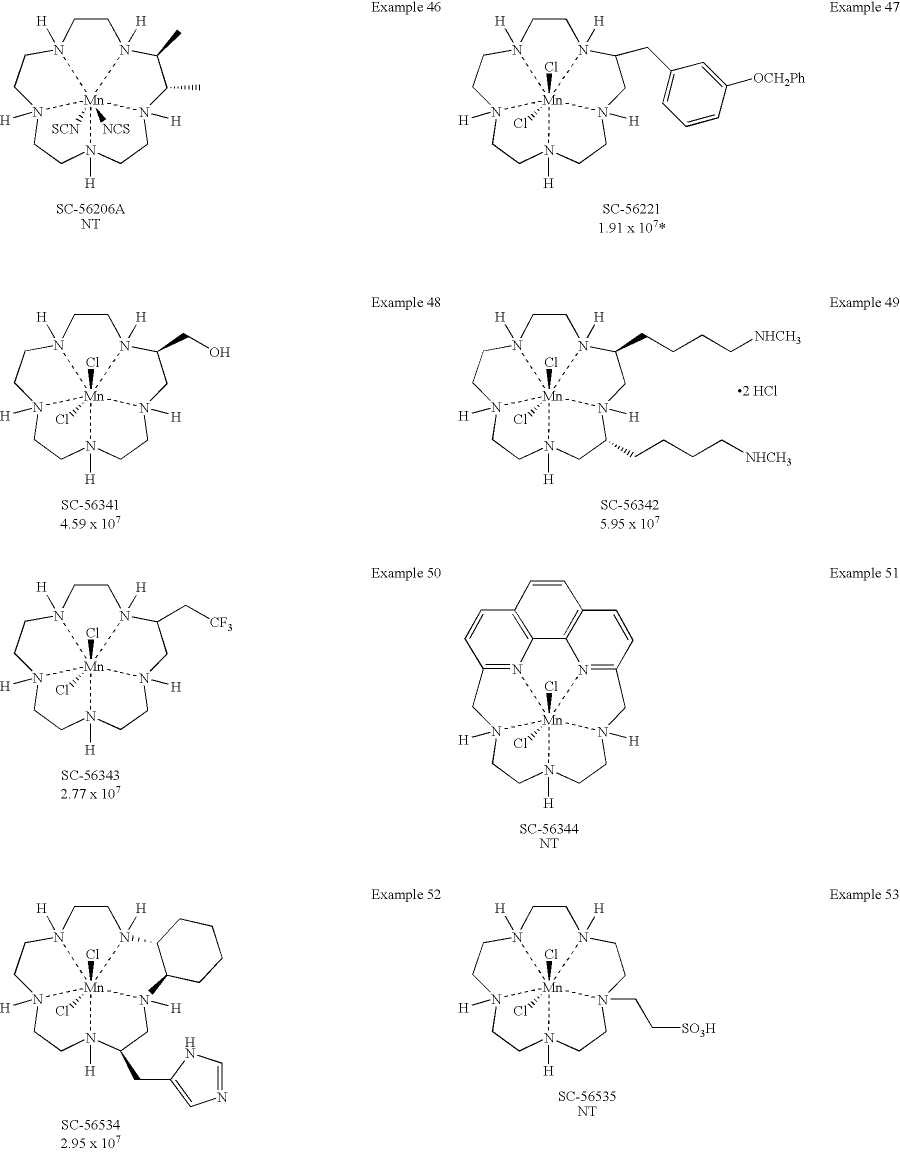

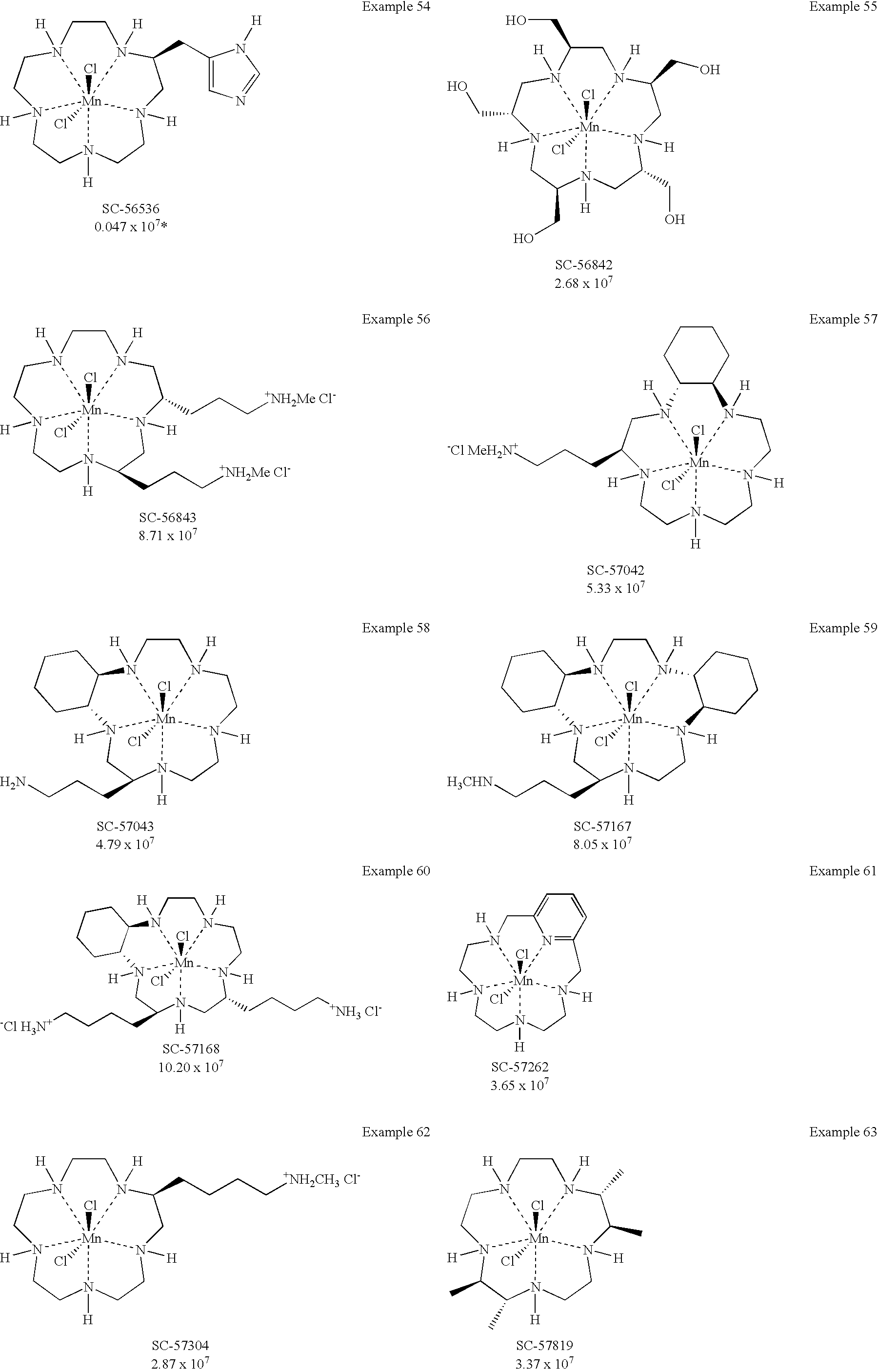

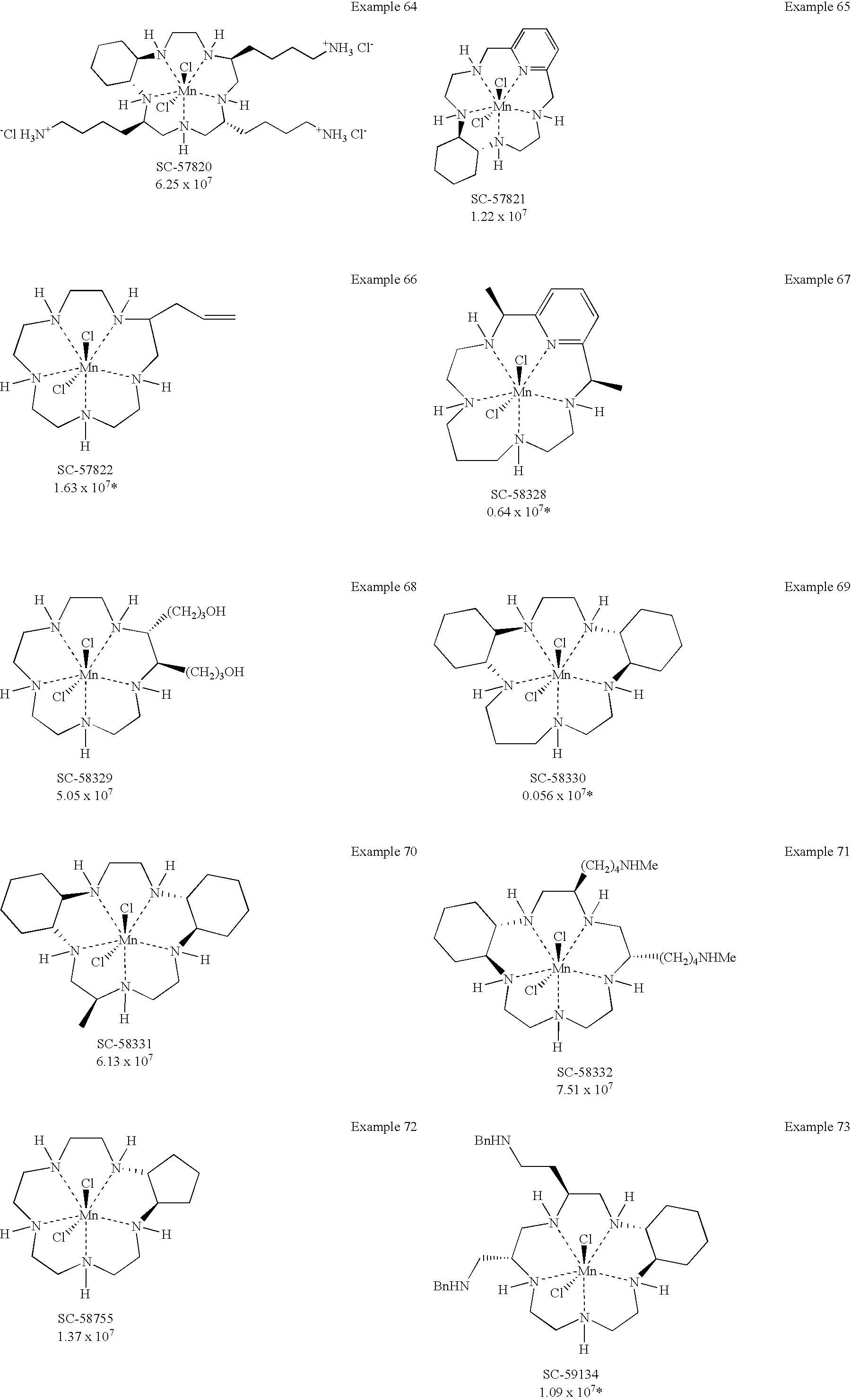

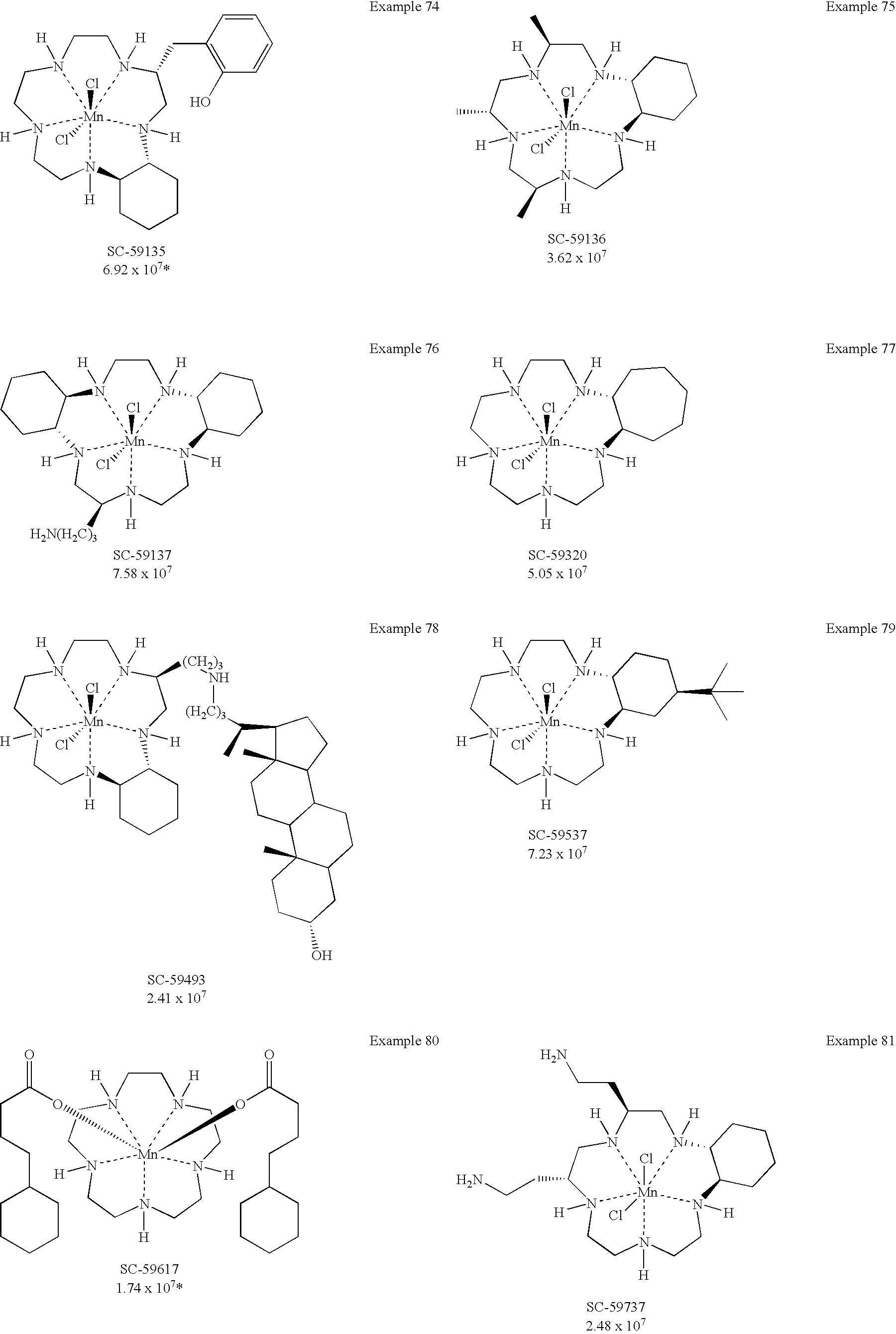

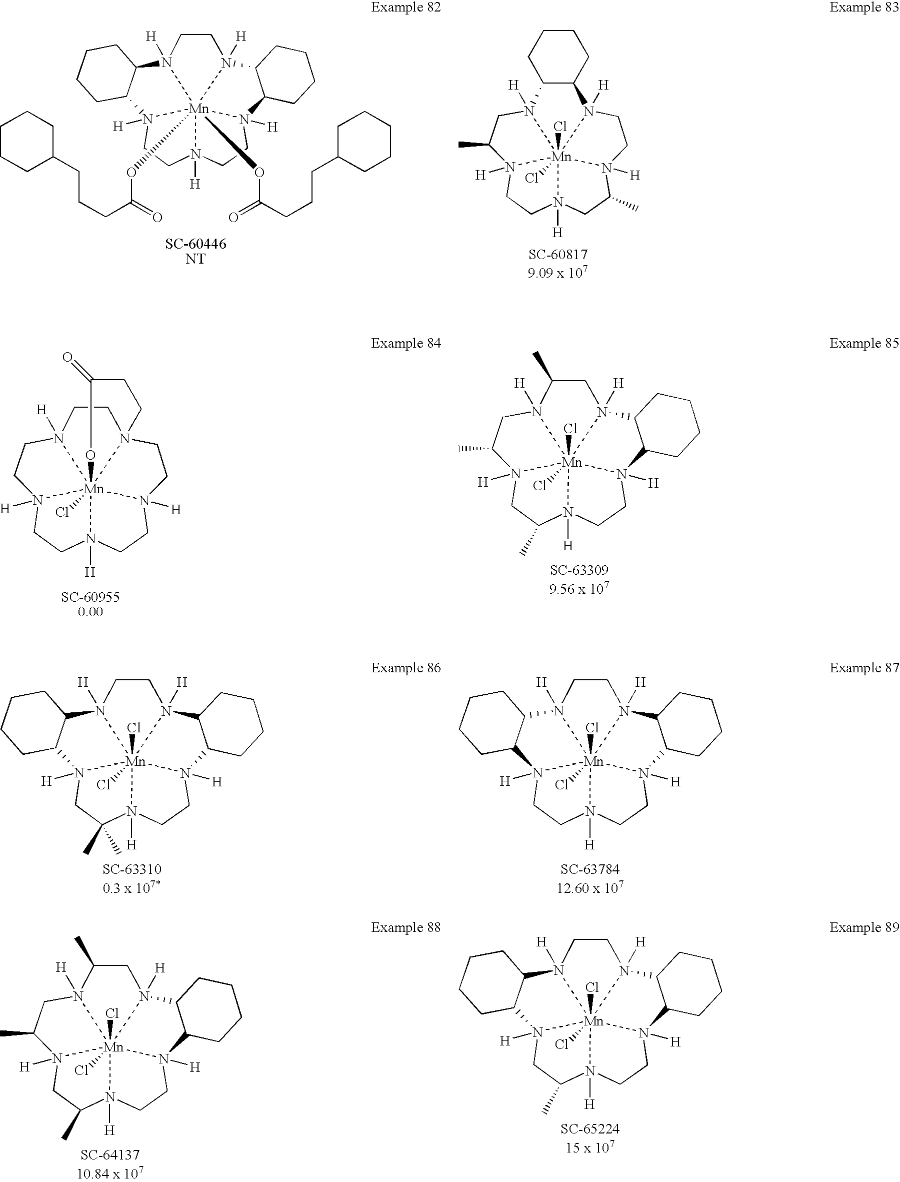

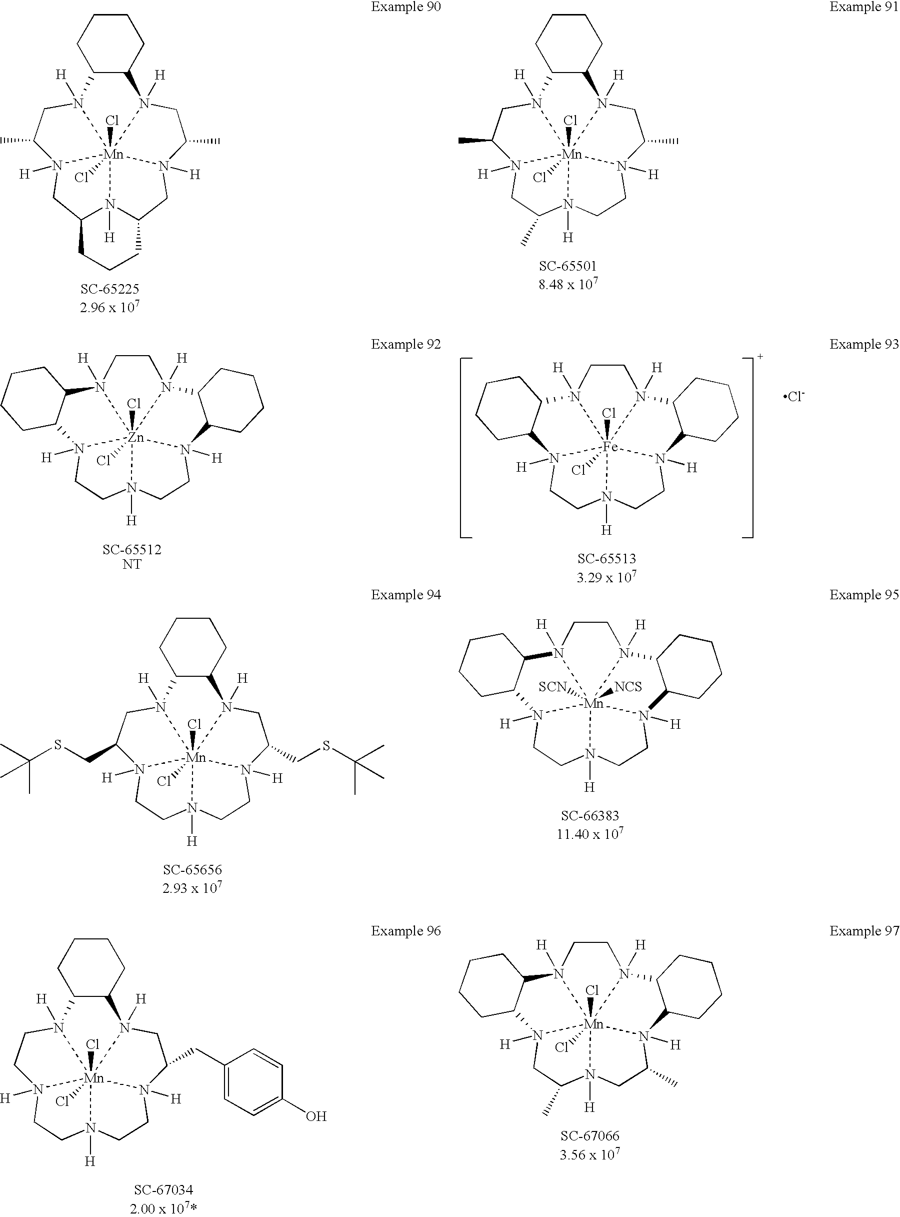

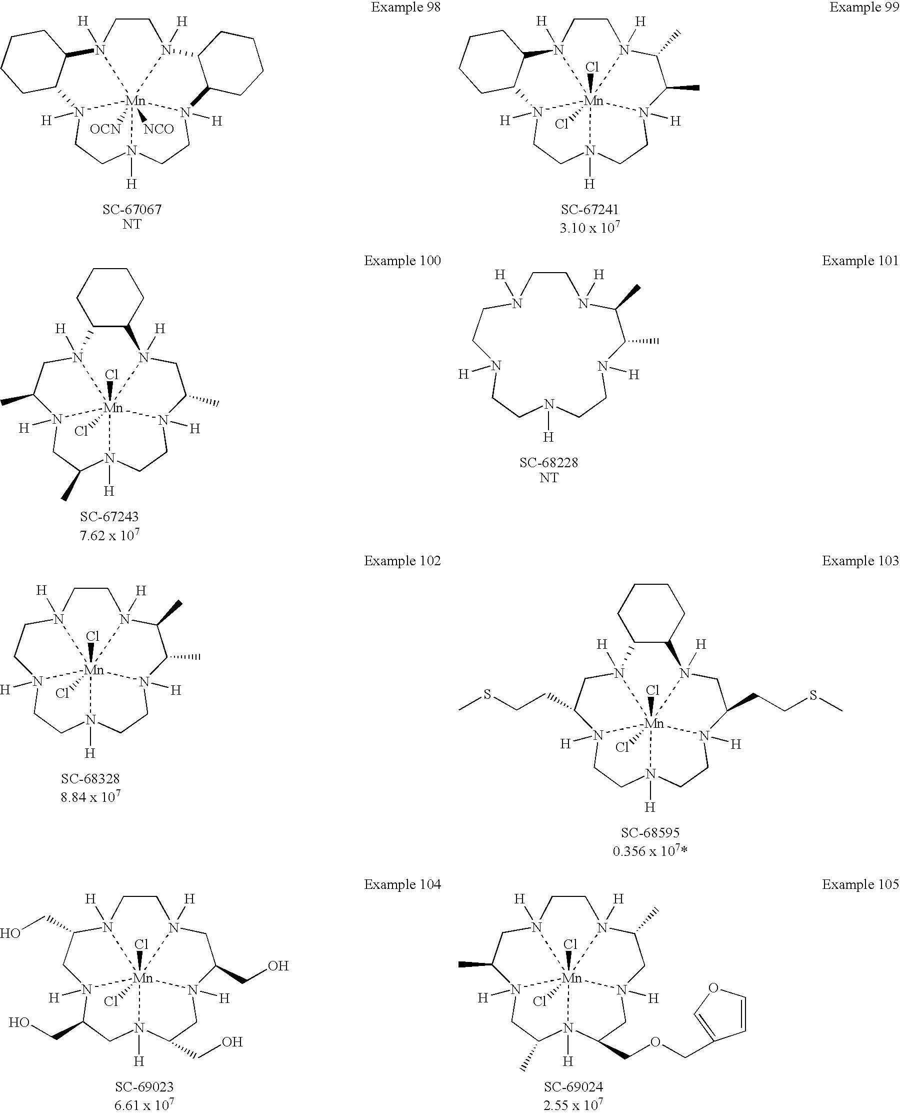

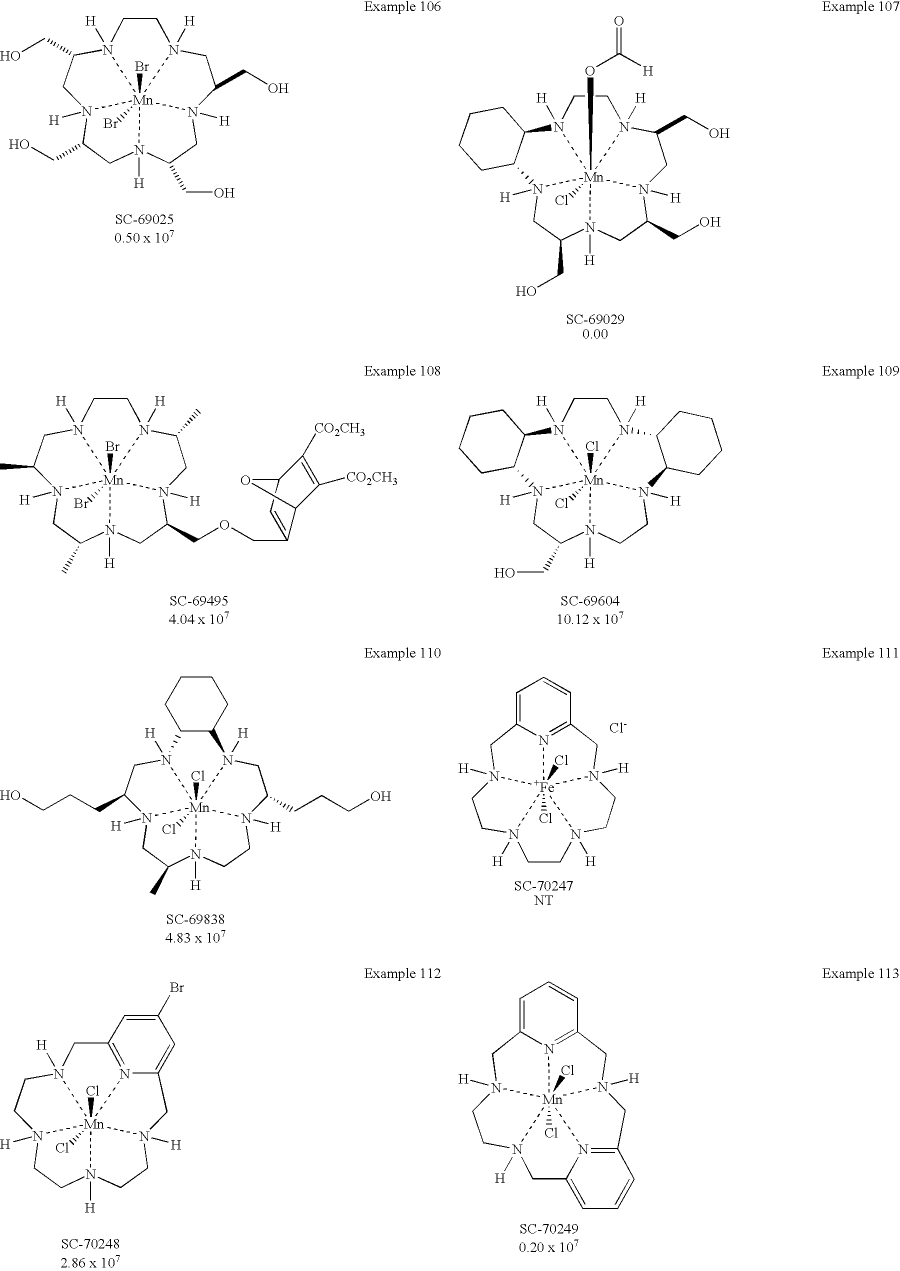

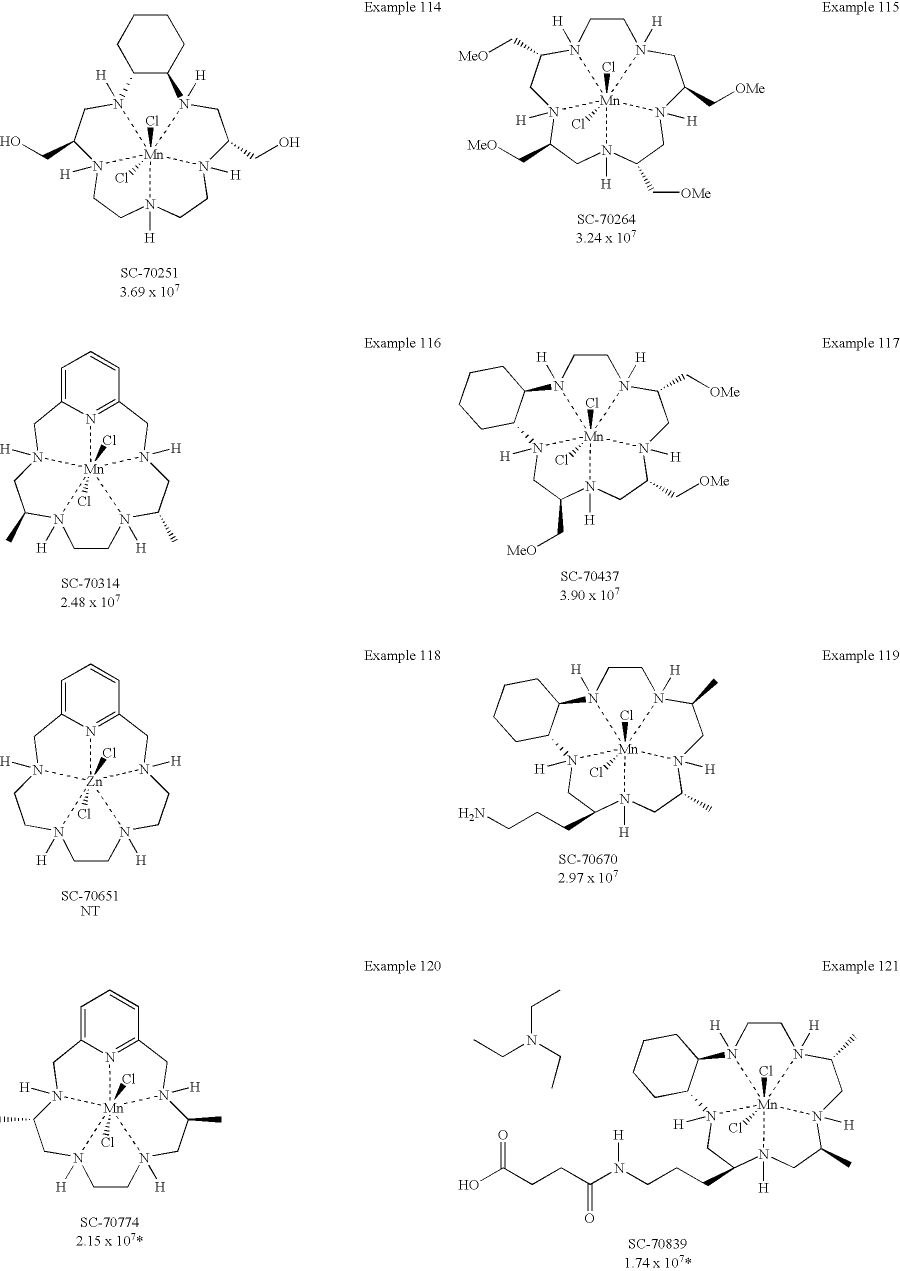

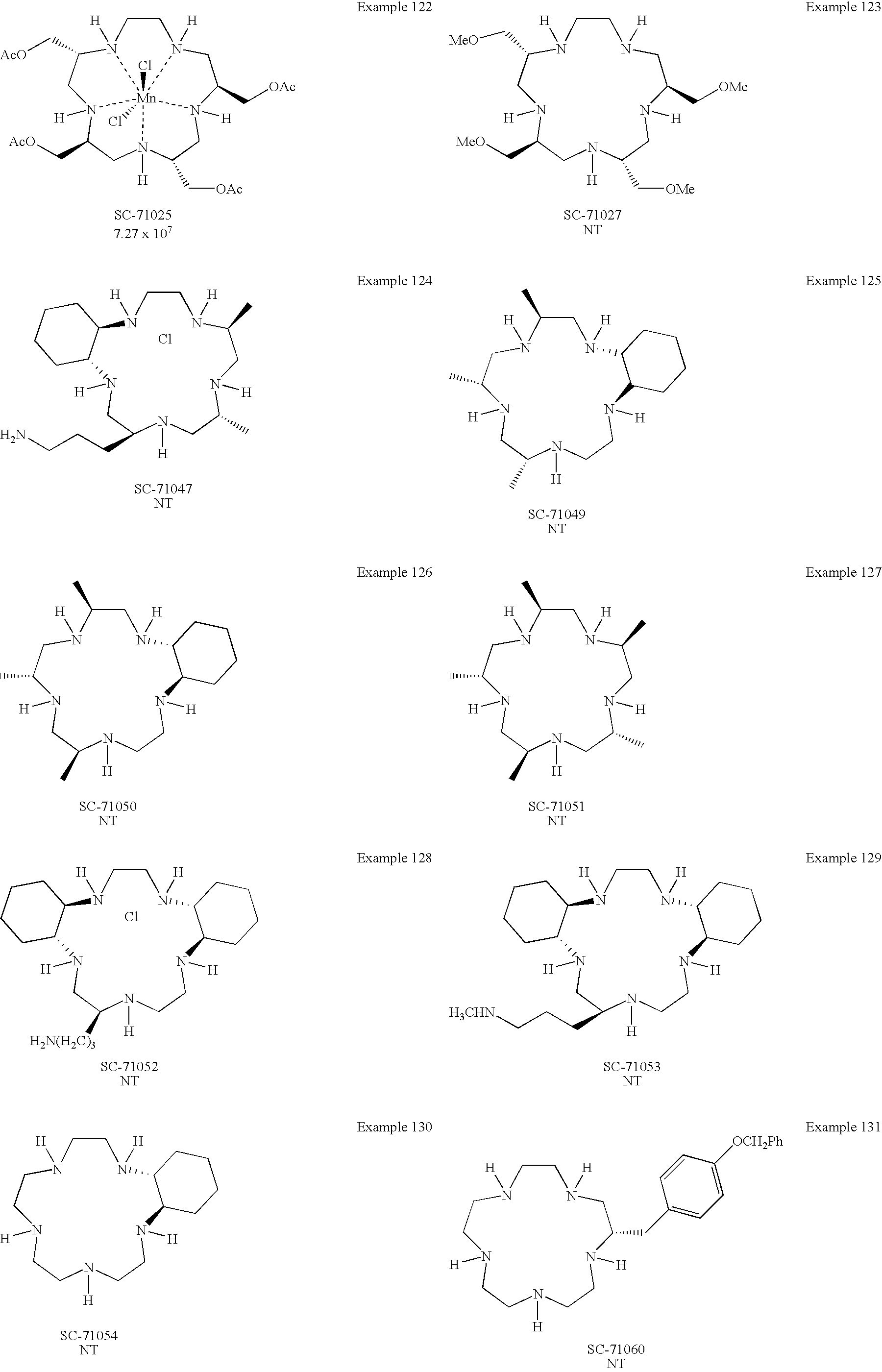

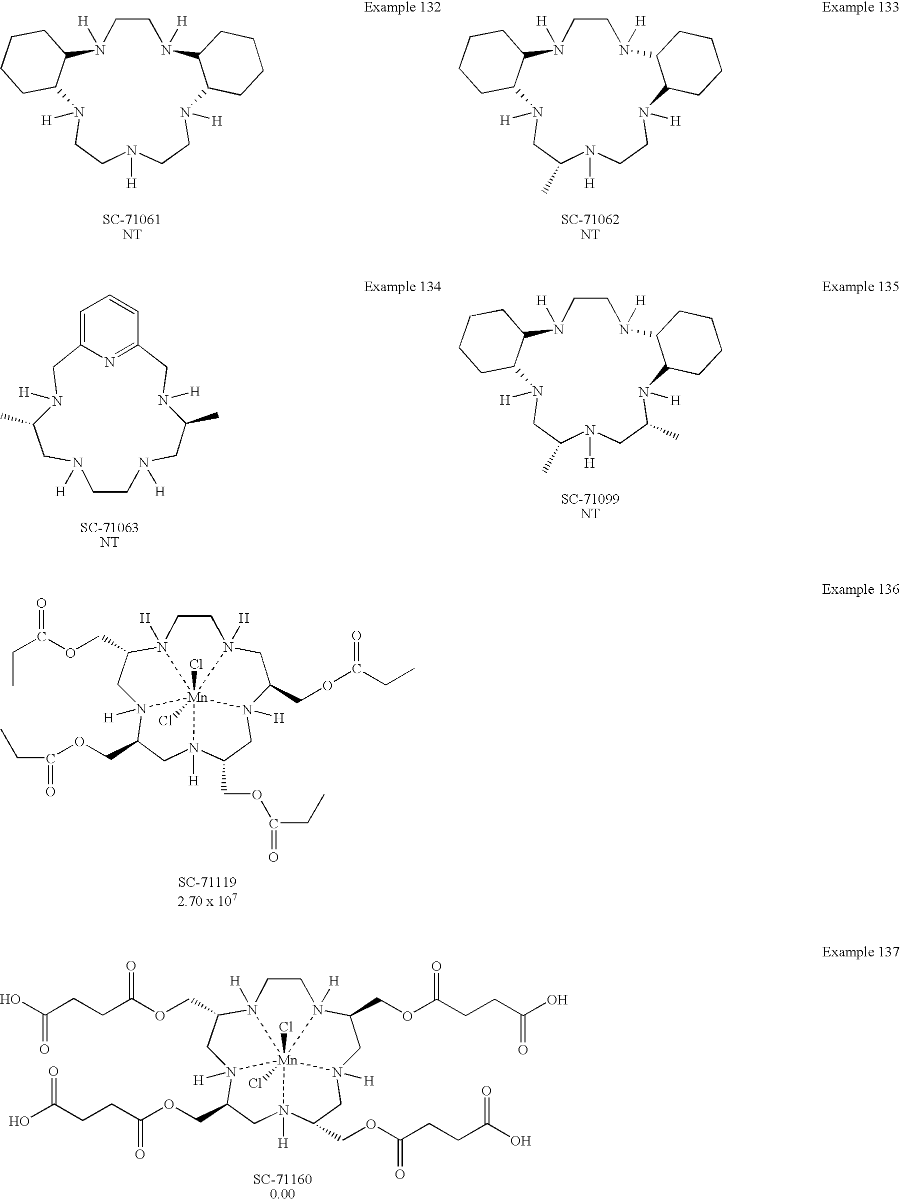

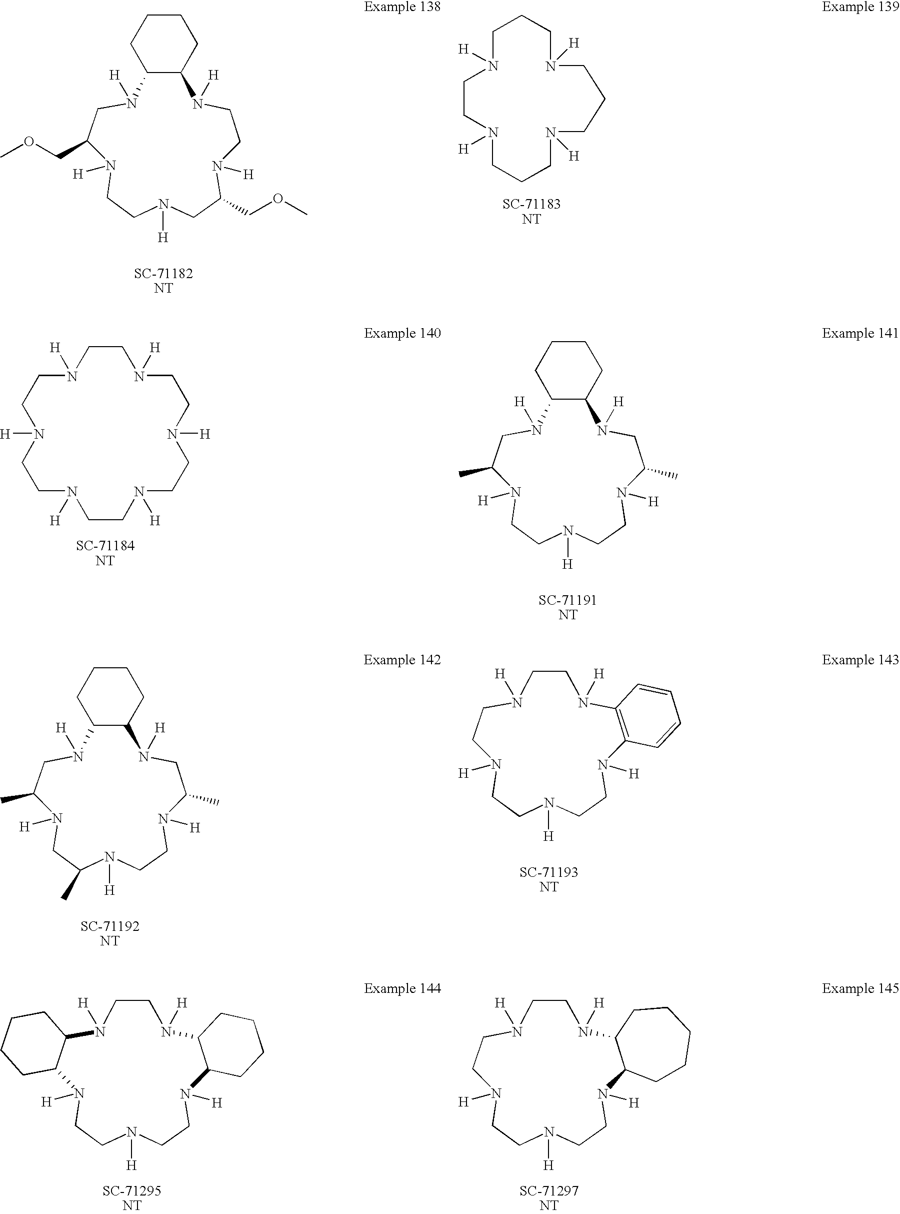

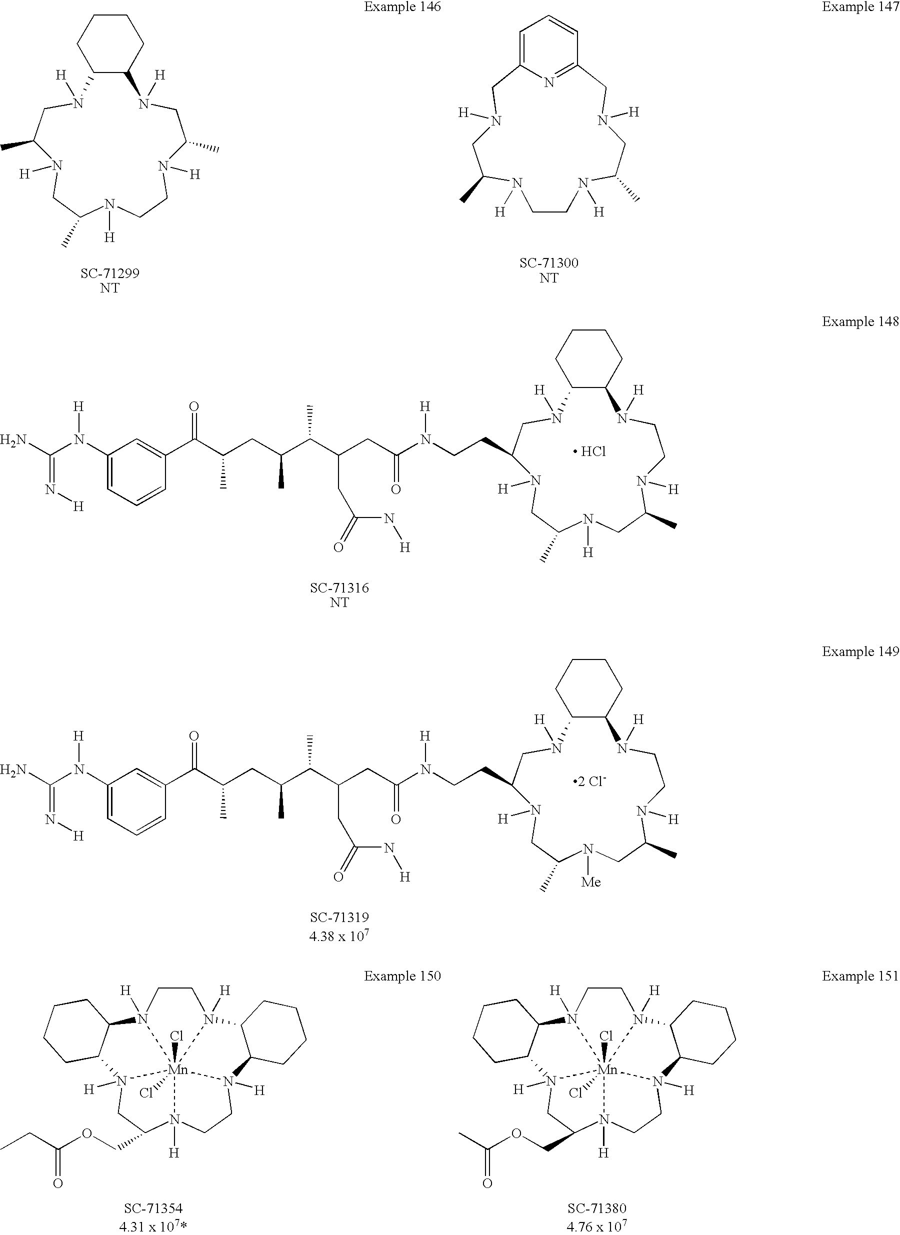

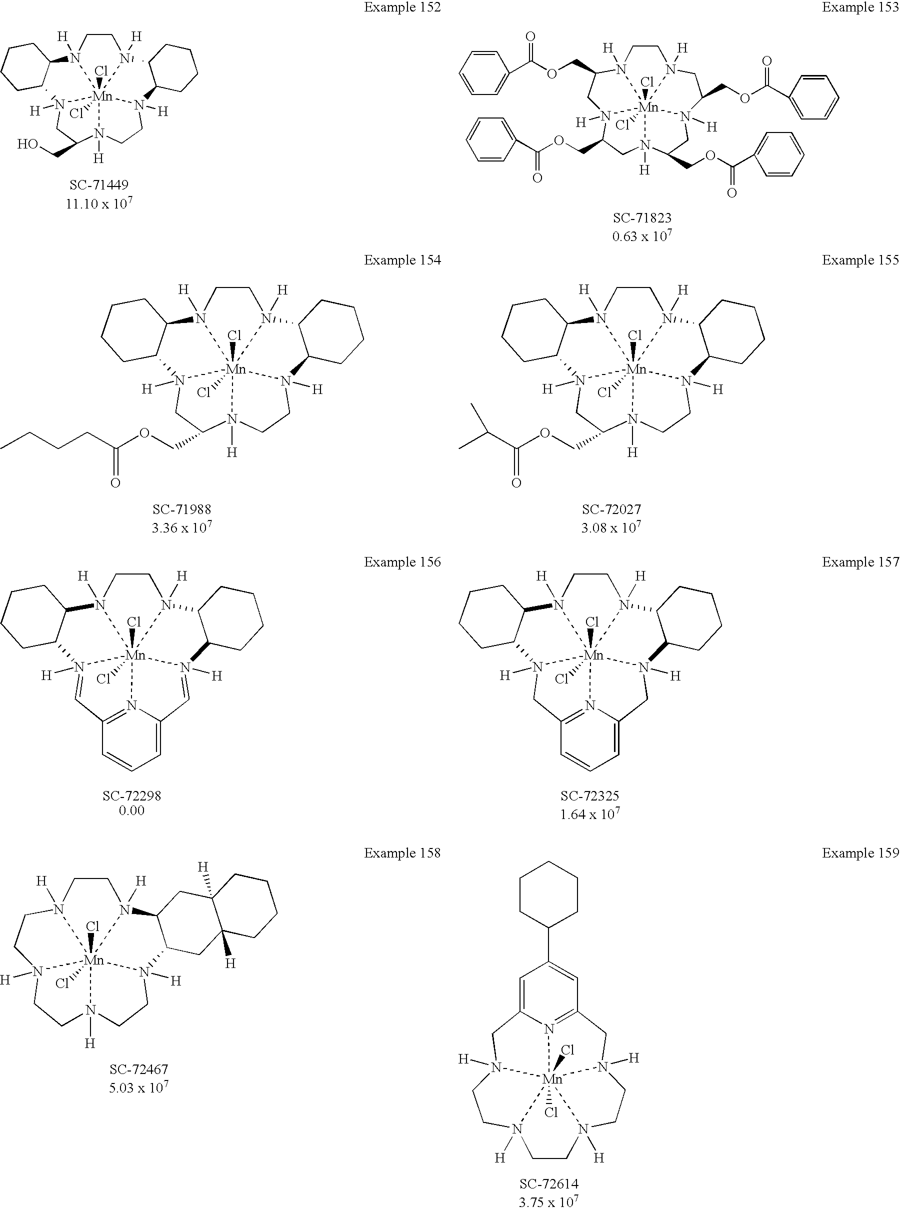

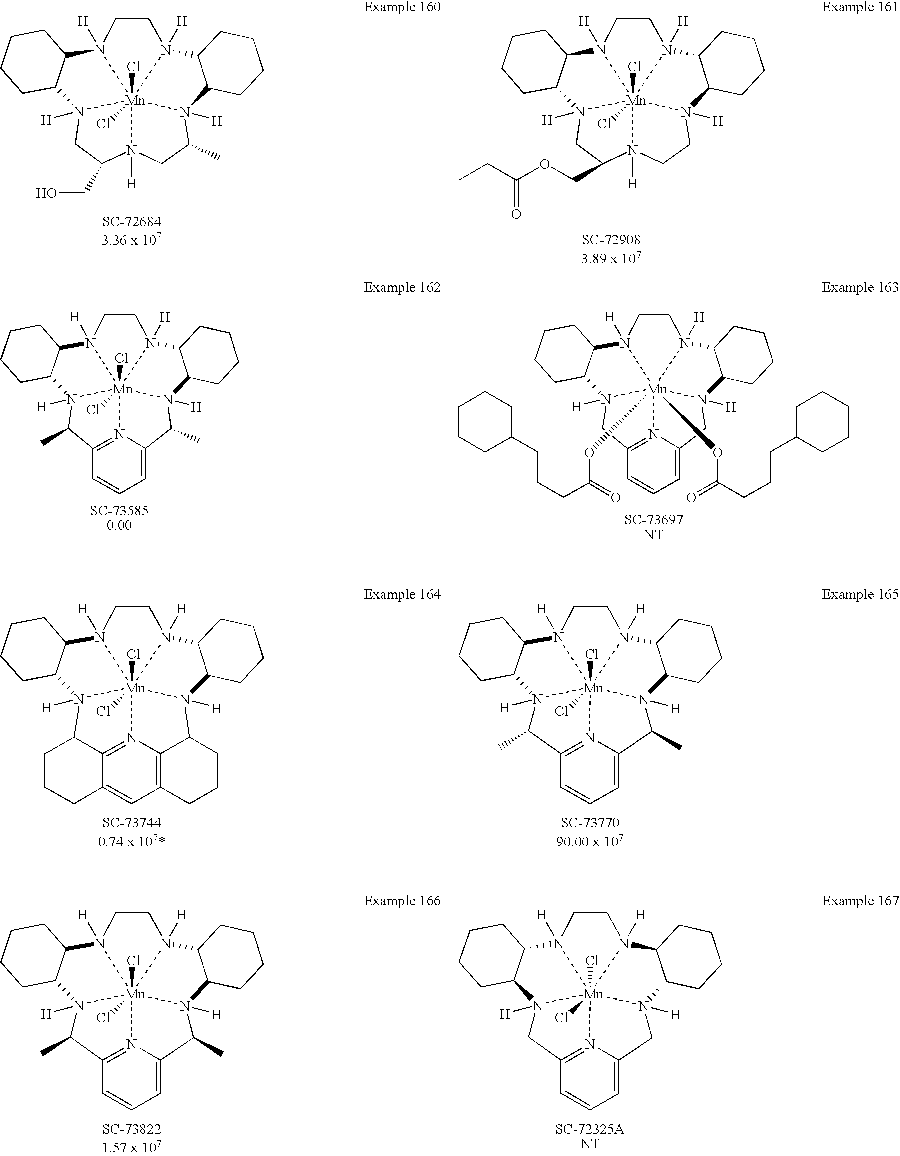

In Examples 168-171 below, male Sprague-Dawley rats were used and all drugs were dissolved in 26 mM NaHCO₃ buffer (0.218 g NaHCO₃ in 100 ml dH2O; pH=8.1 to 8.3) and injections were given subcutaneously (hereinafter “s.c.”). When drug combinations were employed, each drug was injected separately, but concurrently. Drugs employed morphine sulfate and SC-72325A, Example 167 which is an enantiomer of SC-72325 also depicted above. In some studies ketorolac was also used and was given by s.c. injection.

Thermal hyperalgesia and antinociception were assessed in the testing of SC-72325A for treatment of pain. Thermal hyperalgesia was determined by the method of Hargreaves et al., Pain, 32:77-88 (1988). A radiant heat source was focused onto the plantar surface of the affected paw of nerve-injured or carageenan-injected rats. When the animal withdrew its paw, a motion sensor halted the stimulus and timer. A maximal cut-off of 40 seconds was utilized to prevent tissue damage. Paw withdrawal latencies were thus determined to the nearest 0.1 seconds. Reversal of thermal hyperalgesia was indicated by a return of the paw withdrawal latencies to the pre-tremeant baseline latencies (i.e., 21 seconds). Antinociception was indicated by a significant (p≦0.05) increase in paw withdrawal latency above this baseline. Data were converted to % antihyperalgesia or % antinociception by the formula:

100×(test latency—baseline)/(cut-off baseline)

where cut-off was 21 seconds for determining antihyperalgesia and 40 seconds for determining antinociception.

Dose response curves were generated for each drug and drug combination for data obtained at the time of peak effect, which was consistently at the 30 minute time point.

Studies employing combinations of drugs were analyzed for additive or synergistic interactions by isobolographic analysis as described by Tallarida (Tallarida et al., Life Sciences, 45:947-61, 1987) and employed by other (Ossipov et al., J. Pharmacol. Exp. Ther., 255:1107-1116, 1990; Porreca et al., Euro. J. Pharm., 179: 463-468, 1990) by means of a customized Visual Basic computer program (Ossipov, personal communication). Log dose-response curves for each component administered alone were established and the A₅₀ (95% C.L.) were calculated.

Using these methods, the amount of synergy of a combination of compositions can be determined. The preferred combinations of the present invention treat pain using a smaller dose of an analgesic, such as an NSAID or opioid, when compared to administering the analgesic alone. In other words, a preferred combination will result, for example, in the same amount of pain relief after administering 50 mg of an NSAID or opioid in combination with 50 mg of a synthetic superoxide dismutase catalyst as would normally result from administering 500 mg of an NSAID or opioid alone or 500 mg of a synthetic superoxide dismutase catalyst alone.

Conversely, the preferred combinations of the present invention treat pain to a greater extent when compared to treating pain with an analgesic alone or a synthetic superoxide dismutase catalyst alone. In other words, a preferred combination will result, for example, in an equivalent amount of pain relief after administering 500 mg of an NSAID or opioid in combination with 50 mg of a synthetic superoxide dismutase catalyst as would normally result from administering 1,000 mg of the NSAID or opioid or 1,000 mg of a synthetic superoxide dismutase catalyst alone.

Thus, preferred combinations result in additive or synergistic antihypertensive or antinociceptive effects allowing an NSAID or opioid to be administered in a dosage that is at least 50% less than the same NSAID or opioid administered alone. More preferably, the NSAID or opioid combination may be administered in a dosage that is at least 25% less than the same NSAID or opioid administered alone to achieve said therapeutic effect. Still more preferably, the NSAID or opioid may be administered in a dosage that is at least 10% less than the same NSAID or opioid administered alone to achieve said therapeutic effect. And still more preferably, the NSAID or opioid may be administered in a dosage that is at least 1% less than the same NSAID or opioid administered alone to achieve said therapeutic effect.

The A₅₀ for the log dose-response curve of a drug mixture at a fixed ratio was calculated in terms of “total dose” administered. For a given drug combination a theoretical A₅₀ exists such that A₅₀ add=A_{50 drug1}×(p₁+Rp₂) where R is the potency ratio of drug 1 to drug 2, p₁ is the proportion of drug 1 in the mixture and p₂ is the proportion of drug 2. Variances and 95% C.L. for the theoretical additive A₅₀ are derived from the variances of each drug administered alone. A t-test is employed to compare the theoretical additive A₅₀ and 95% C.L. to that obtained for the mixture. A significantly ((p <0.05); t-test) lower experimental value compared to theoretical value denotes a synergistic interaction. See Table 1 below.

### Example 168

### SC-72325A Treats Pain

Analgesic effects provided by subcutaneous injection of SC-72325A was studied by formalin-induced hind paw licking response. Male CD-1 mice (Charles River, 28-35 gm) were allowed to feed ad libitum. Mice were housed 5-7 per cage in a temperature controlled room with a twelve hour light-dark cycle. Determination of antinociception was assessed between 7:00 and 10:00 AM. Groups consisted of 7-14 mice, and each animal was used for one experimental condition. The antinociceptive effects of SC-72325A were tested in the formalin-induced hind paw licking procedure (Hunskaar et al., Pain, 30: 103-114, 1987). Mice were injected with by sub-plantar administration with formalin (20 μg of a 1% stock solution) and the duration of paw licking was monitored in the periods of 5-10 minutes (Phase I) and 15-30 minutes (Phase II) thereafter. SC-72325A (10 mg/kg) was given s.c. 10 minutes prior to formalin.

At 10 mg/kg, the s.c. injection of SC-72325A had a small inhibitory effect on phase 1 of the response but nearly completely abolished Phase II of the response. See FIG. 6.

### Example 169

### Antihyperalgesia and Antinociception Synergy Using SC-72325A and Morphine Combination

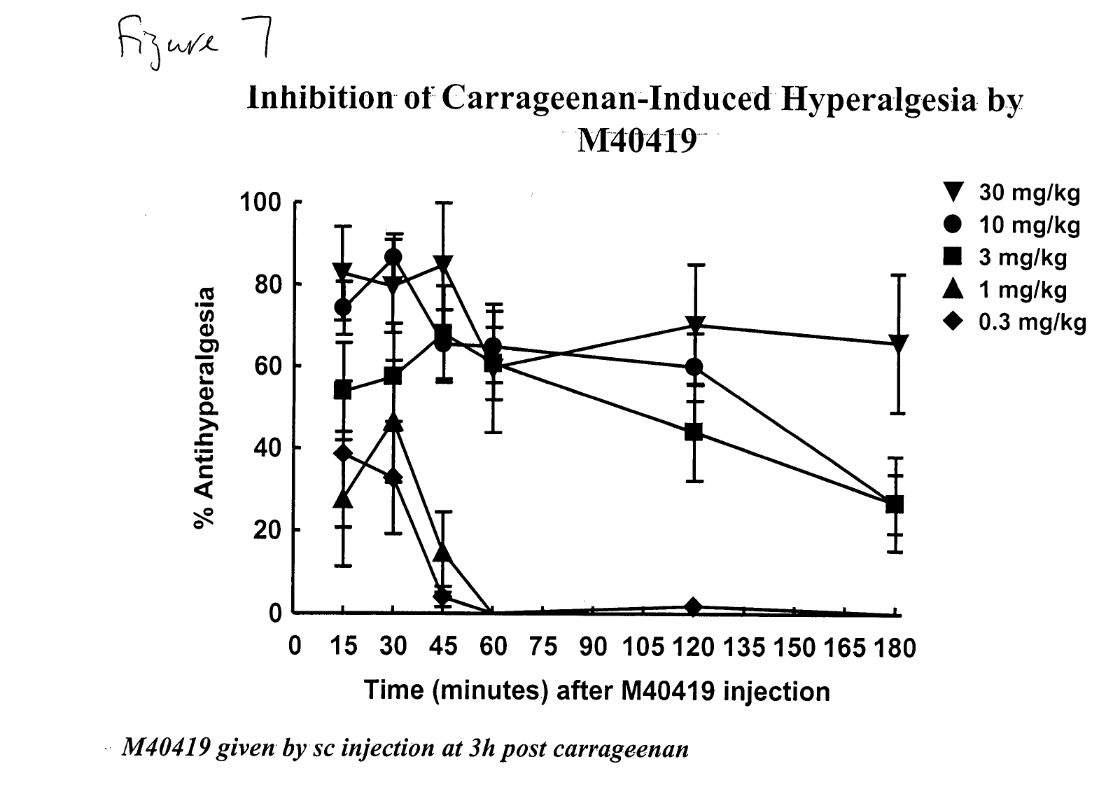

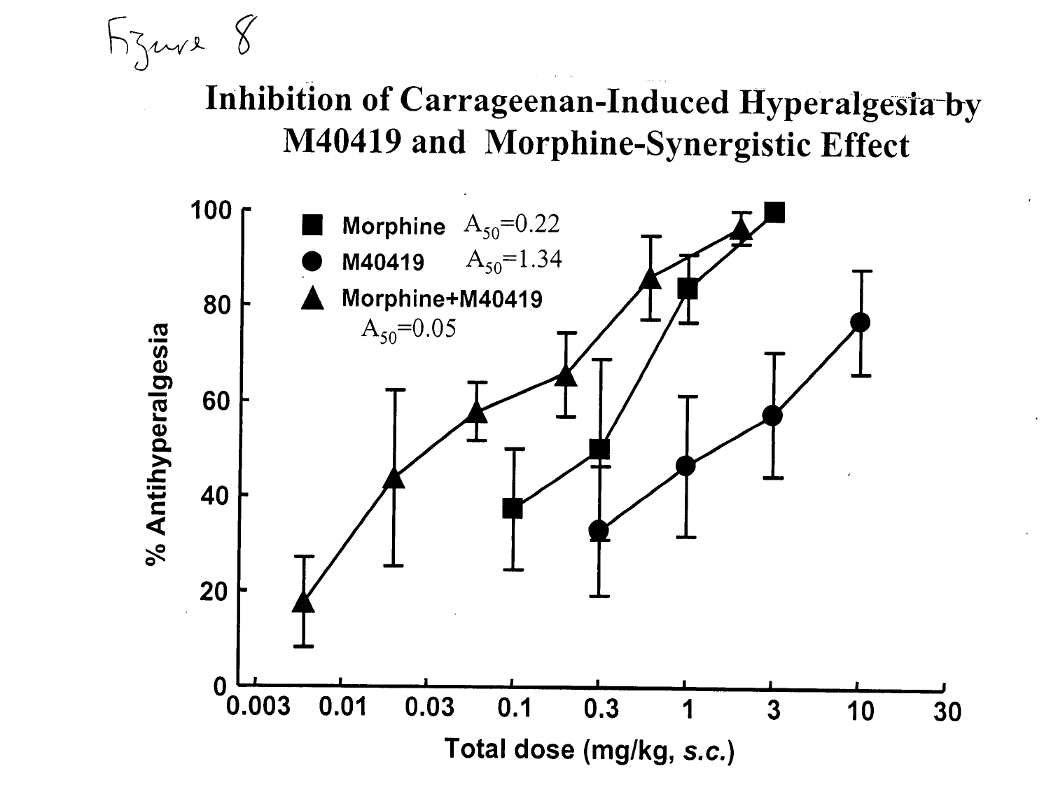

Carrageenan-induced inflammation is a well characterized and commonly employed model of peripheral inflammation. This procedure reliably produces a marked inflammatory response within 3 hours of injection which is indicated by swelling of the hind paw, edema, rubor and hyperalgesia and allodynia (Kocher et al., 1987, Ossipov et al., 1995). Peripheral inflammation was induced in the hind paw of male Sprague-Dawley rats by injecting 0.1 ml of a 2% λ-carrageenan suspension into the subplanar surface of the hind paw of lightly ether-anesthetized rats. All drugs were prepared according to the methods set forth in Example 6, above. Testing was performed 15, 30, 45, 60, 120 and 180 minutes after drug injections. The s.c. injection of SC-72325A produced time-related and dose-dependant antihyperalgesia over the dose range of 0.3 to 30 mg/kg. See FIG. 7. Similarly, morphine also produced time-related and dose-dependent antihyperalgesia and antinociception over the dose range of 0.03 to 10 mg/kg. The 1:1 combination of SC-72325A with morphine produced antihyperalgesia activities at much lower doses than either drug alone. See FIG. 8. Isobolographic analyses revealed that the combination of SC-72325A with morphine resulted in a definitive synergistic interaction against hyperalgesia; the A₅₀ values with confidence intervals are presented in Table 1, below. The antihyperalgesic A₅₀ value for the 1:1 combination of SC-72325A plus morphine was 0.046 mg/kg, s.c., which was significantly (p≦0.05) less than the calculated theoretical A₅₀ value for the combination if the activity was additive. See Table 1. SC-72325A also exhibited a slight antinociceptive effect.

TABLE 1

| Antihyperalgesia | A₅₀ (mg/kg, s.c.) |
|---|---|
| SC-72325A | 1.34 |
| Morphine | 0.22 |
| Morphine + SC-72325A | 0.046 |
| Theoretical Additive Curve | 0.380 |

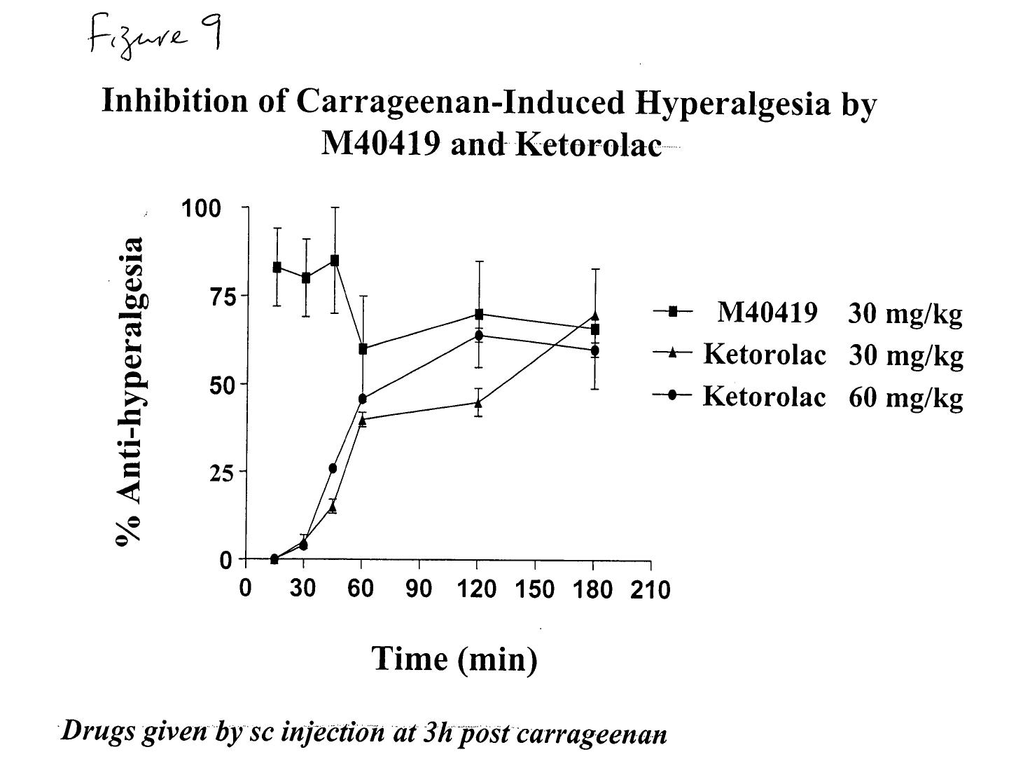

FIG. 9 shows that the onset of action SC-72325A was much faster than the one obtained with ketorolac. In fact, when compared to ketorolac, SC-72325A was more potent and more efficacious in this model.

### Example 170

### SC-72325A Inhibition of Neuropathic Pain

Neuropathic pain (L₅/L₆ SNL) was also utilized to assess the antinociceptive effects of SC-72325A. Nerve ligation injury was performed according to the method described by Kim and Chung (1992). This technique reliably produces signs of clinical neuropathic dysesthesias, including tactile allodynia, thermal hyperalgesia and behavior suggestive of spontaneous pain. Rats were anesthetized with 2% halothane in O₂ delivered at 2 liters/minute. The skin over the caudal lumber region was incised and the muscles retracted. The L₅ and L₆ spinal nerves were exposed, carefully isolated, and tightly ligated with 4-0 silk suture to the dorsal root ganglion. After ensuring homeostatic stability, the wounds were sutured, and the animals allowed to recover in individual cages. Any rats exhibiting signs of motor deficiencies were euthanized. Testing was performed 15, 30, 45, 60 and 90 minutes after drug injections.

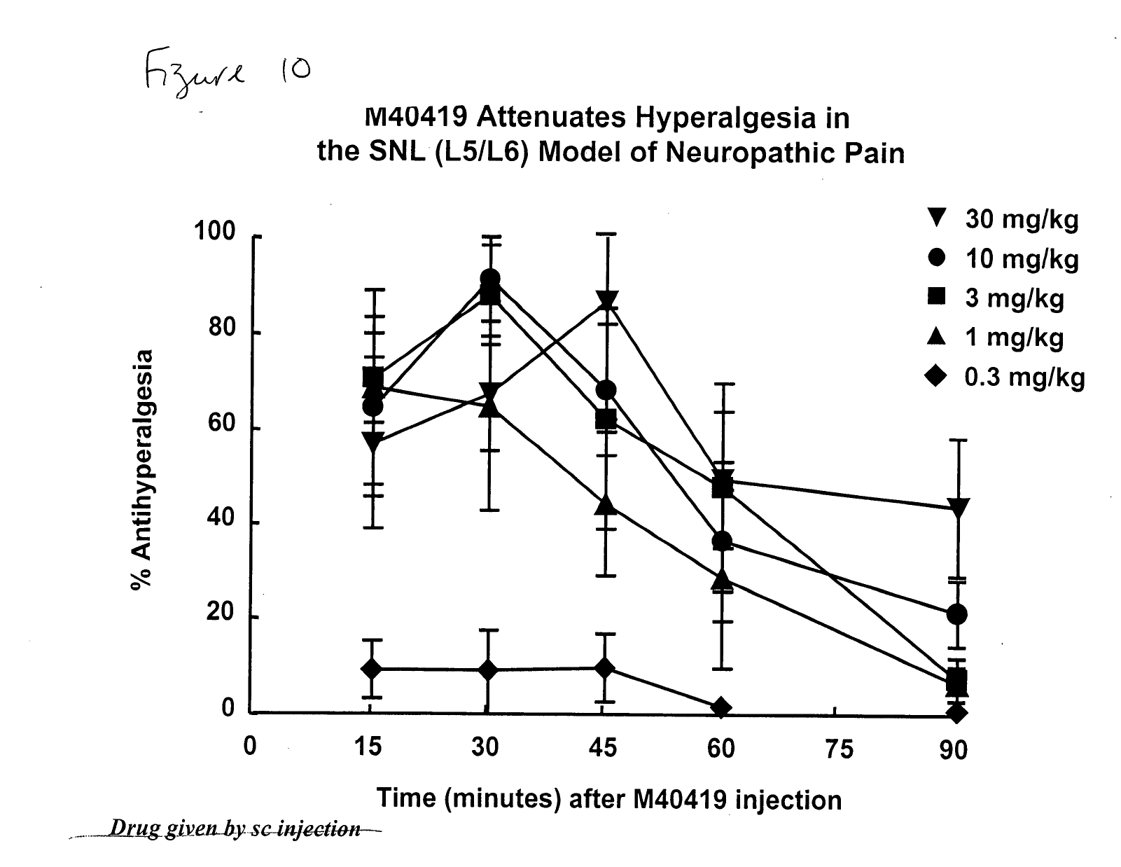

The s.c. injection of SC-72325A produced time-related and dose-dependent antihyperalgesia over the dose range of 1 to 30 mg/kg. See FIG. 10. One of the highest doses tested, 10 mg/kg, produced an antihyperalgesic effect of 91±8.8% MPE and an antinociceptive effect of 39 ±6.4% MPE 30 minutes after injection. SC-72325A also exhibited a slight antinociceptive effect.

### Example 171

### SC-72325A Inhibition of Allodynia

Chronic constriction injury was performed as described by Bennett and Xie (1988). Male Sprague-Dawley rats were lightly anesthetized and the sciatic nerve isolated and exposed. Four chronic gut ligatures (4-0) are loosely placed around the nerve about 1 to 2 mm apart and the wound closed. Signs of hyperalgesia and spontaneous pain, including guarding of the hind paw and spontaneous nocifensive responses are normally present within 4 days of surgery. Any rats exhibiting signs of motor deficiency were euthanized. Cold allodynia was evaluated by placing rats in a shallow pan of ice water (0° C., 3 cm deep). The response latency to withdrawal of the hind paw or escape behavior is measured. Normal or sham-operated rats typically show no response during the 30 second exposure to the ice water. Testing was performed 15, 30, 45 and 60 minutes after drug injections. Drugs were given by s.c. injection.

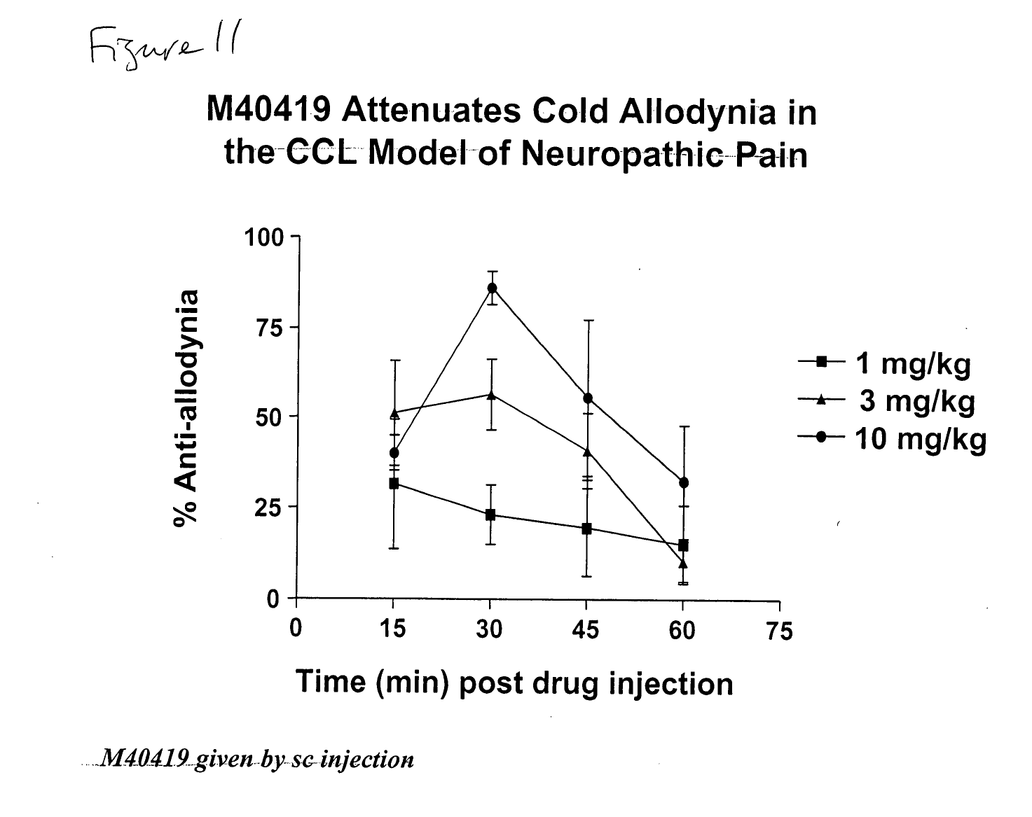

The s.c. injection of SC-72325A produced time-related and dose-dependent attenuation of cold allodynia over the dose range of 1 to 10 mg/kg. See FIG. 11.

Other Embodiments

The detailed description set-forth above is provided to aid those skilled in the art in practicing the present invention. However, the invention described and claimed herein is not to be limited in scope by the specific embodiments herein disclosed herein because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A combination comprising:
(a) at least one opioid; and
(b) at least one synthetic superoxide dismutase catalyst.

2. A combination according to claim 1, wherein the combination is capable of treating, preventing, reversing or inhibiting pain or inflammation when administered to a patient in need thereof.

3. A combination according to claim 2, wherein the combination is capable of producing an additive or synergistic antihyperalgesia or antinociception effect in the patient after administering the combination.

4. A combination according to claim 3, wherein the opioid of the combination comprises at least about 50% less than the same opioid administered alone to achieve the antihyperalgesia or antinociception effect.

5. A combination according to claim 4, wherein the opioid of the combination comprises at least about 25% less than the same opioid administered alone to achieve the antihyperalgesia or antinociception effect.

6. A combination according to claim 5, wherein the opioid of the combination comprises at least about 10% less than the same opioid administered alone to achieve the antihyperalgesia or antinociception effect.

7. A combination according to claim 6, wherein the opioid of the combination comprises at least about 1% less than the same opioid administered alone to achieve the antihyperalgesia or antinociception effect.

8. A combination according to claim 2, wherein the opioid and the synthetic superoxide dismutase catalyst are combined prior to administration to the patient.

9. A combination according to claim 2, wherein the opioid and the synthetic superoxide dismutase catalyst are combined upon administration to the patient.

10. A combination according to claim 1, wherein the opioid is selected from the group consisting of morphine, oxycontin, oxycodone, codeine, fentanyl and any combination thereof.

11. A combination according to claim 1, wherein the synthetic superoxide dismutase catalyst is represented by the formula: 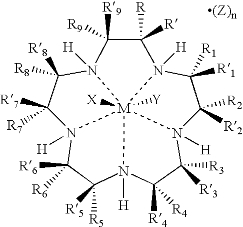
wherein
(a) R, R′, R₁, R′₁, R₂, R′₂, R₃, R′₃, R₄, R′₄, R₅, R′₅, R₆, R′₆, R₇ R′₇, R₈, R′₈, R₉, and R′₉ independently are selected from the group consisting of hydrogen and substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and
(b) optionally, R₁ or R′₁ and R₂ or R′₂, R₃ or R′₃ and R₄ or R′₄, R₅ or R′₅ and R₆ or R′₆, R₇ or R′₇ and R₈ or R′₈, or R₉ or R′₉ and R or R′ together with the carbon atoms to which they are attached independently form a substituted or unsubstituted, saturated, partially saturated or unsaturated cyclic or heterocyclic having 3 to 20 carbon atoms; and
(c) optionally, R or R′ and R₁ or R′₁, R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle wherein the hydrogen attached to the nitrogen which is both part of the heterocycle and the macrocycle and the R groups attached to the carbon atoms which are both part of the heterocycle and the macrocycle are absent; and
(d) optionally, R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a substituted or unsubstituted, saturated, partially saturated, or unsaturated cyclic or heterocyclic having 3 to 20 carbon atoms; and
(e) optionally, one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉, attached to a different carbon atom in the macrocycle are bound to form a strap represented by the formula:
—(CH₂)ₓ—M—(CH₂)_{w}—L—(CH₂)_{z}—J—(CH₂)_{y—}
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and
(f) combinations of any of (a) through (e) above; and
wherein
M is selected from the group consisting of copper, manganese and zinc;
X, Y and Z are pharmaceutically acceptable counter ions, or together are a pharmaceutically acceptable polydentate ligand; and
n is an integer from 0 to 3.

12. A combination according to claim 11, wherein the synthetic superoxide dismutase catalyst is represented by the formula:

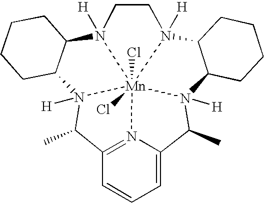

13. A combination according to claim 11, wherein the synthetic superoxide dismutase catalyst is represented by the formula:

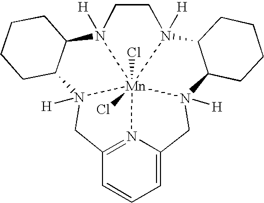

14. A combination according to claim 11, wherein the synthetic superoxide dismutase catalyst is represented by the formula:

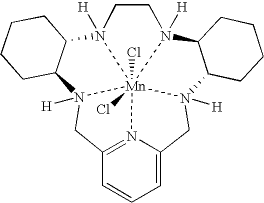

15. A compound of the formula Aₙ-Qₘ, wherein A is a superoxide dismutase catalyst moiety, Q is an opioid moiety, and n and m are independently integers from 1 to 3.

16. A compound according to claim 15, wherein the opioid moiety is selected from the group consisting of morphine, oxycontin, oxycodone, codeine, fentanyl and any combination thereof.

17. A compound according to claim 15, wherein the synthetic superoxide dismutase catalyst moiety is represented by the formula: 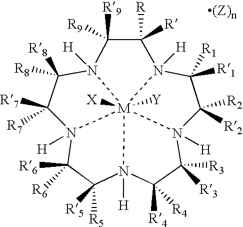
wherein
(a) R, R′, R₁, R′₁, R₂, R′₂, R₃, R′₃, R₄, R′₄, R₅, R′₅, R₆, R′₆, R₇, R′₇, R₈, R′₈, R₉, and R′₉ independently are selected from the group consisting of hydrogen and substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and
(b) optionally, R₁ or R′₁, and R₂ or R′₂, R₃ or R′₃ and R₄ or R′₄, R₅ or R′₅ and R₆ or R′₆, R₇ or R′ ₇ and R₈ or R′₈, or R₉ or R′₉ and R or R′ together with the carbon atoms to which they are attached independently form a substituted or unsubstituted, saturated, partially saturated or unsaturated cyclic or heterocyclic having 3 to 20 carbon atoms; and
(c) optionally, R or R′ and R₁ or R′₁, R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle wherein the hydrogen attached to the nitrogen which is both part of the heterocycle and the macrocycle and the R groups attached to the carbon atoms which are both part of the heterocycle and the macrocycle are absent; and
(d) optionally, R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a substituted or unsubstituted, saturated, partially saturated, or unsaturated cyclic or heterocyclic having 3 to 20 carbon atoms; and
(e) optionally, one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ attached to a different carbon atom in the macrocycle are bound to form a strap represented by the formula:
—(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and
(f) combinations of any of (a) through (e) above; and
wherein
M is selected from the group consisting of copper, manganese and zinc;
X, Y and Z are pharmaceutically acceptable counter ions, or together are a pharmaceutically acceptable polydentate ligand; and
n is an integer from 0 to 3.

18. A compound according to claim 17, wherein the synthetic superoxide dismutase catalyst is represented by the formula:

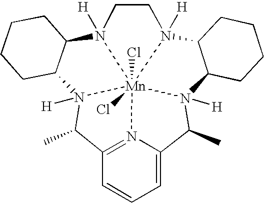

19. A compound according to claim 17, wherein the synthetic superoxide dismutase catalyst is represented by the formula:

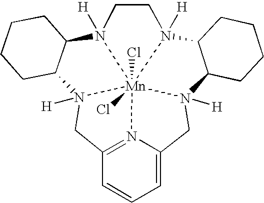

20. A compound according to claim 17, wherein the synthetic superoxide dismutase catalyst is represented by the formula: [chemical structure]
